(19) United States
(12) Patent Application Publication
(10) Pub. No.: US 2015/0293104 A1
(43) Pub. Date: Oct. 15, 2015

(54) EPITHELIAL OVARIAN CANCER DIFFERENTIATION MARKER

(71) Applicants: Hirofumi Nozaki, Ibaraki (JP); Takashi Ohkura, Ibaraki (JP); Atsushi Kuno, Ibaraki (JP); Maki Sogabe, Ibaraki (JP); Tomomi Kubota, Ibaraki (JP); Hiroyuki Kaji, Ibaraki (JP); Akira Togayachi, Ibaraki (JP); Yuzuru Ikehara, Ibaraki (JP); Hisashi Narimatsu, Ibaraki (JP); Hayao Nakanishi, Nagoya-shi -Aichi (JP); Toru Nakanishi, Nagoya - Shi- Aichi (JP)

(72) Inventors: Hirofumi Nozaki, Ibaraki (JP); Takashi Ohkura, Ibaraki (JP); Atsushi Kuno, Ibaraki (JP); Maki Sogabe, Ibaraki (JP); Tomomi Kubota, Ibaraki (JP); Hiroyuki Kaji, Ibaraki (JP); Akira Togayachi, Ibaraki (JP); Yuzuru Ikehara, Ibaraki (JP); Hisashi Narimatsu, Ibaraki (JP); Hayao Nakanishi, Nagoya-shi -Aichi (JP); Toru Nakanishi, Nagoya - Shi- Aichi (JP)

(73) Assignee: National Institute of Advanced Industrial Science and Technology, Tokyo (JP)

(21) Appl. No.: 14/413,846

(22) PCT Filed: Jul. 12, 2012
(86) PCT No.: PCT/JP2012/067798

§ 371 Date: Jun. 8, 2015

(51) Int. Cl. G01N 33/574

(52) U.S. Cl. CPC G01N 33/57449; G01N 2333/90287; G01N 2333/902; G01N 2333/8121; G01N 2333/96458; G01N 2333/78

(57) ABSTRACT

An object of the present invention is to develop and provide an epithelial ovarian cancer diagnosis marker with which epithelial ovarian cancer can be detected inexpensively, conveniently, and low invasively with high accuracy, and a method for determining the presence or absence of epithelial ovarian cancer using the marker. The present invention provides a glycoprotein having a glycan-linked asparagine residue at a particular site of the glycoprotein secreted from an epithelial ovarian cancer cell, or a fragment thereof having the glycan as an epithelial ovarian cancer diagnosis marker. The present invention also provides a method for determining the presence or absence of epithelial ovarian cancer using the glycoprotein.

### TECHNICAL FIELD

The present invention relates to a glycoprotein for an epithelial ovarian cancer diagnosis marker or a fragment thereof having the glycan, and a method for determining the presence or absence of epithelial ovarian cancer using the marker.

### BACKGROUND ART

Ovarian cancer is a cancer having the second-highest incidence after breast cancer among gynecological cancers. The early detection of ovarian cancer is difficult because of almost no subjective symptom at an early stage in the course of the disease. In many cases, its symptoms have already progressed when found. Hence, the ovarian cancer results in poor prognosis and the highest mortality rate among gynecological cancers.

Ovarian cancer is known to include surface epithelial-stromal tumor (epithelial ovarian cancer), which is developed from the surface epithelial cells of the ovary, germ cell tumor, which is developed from germ cells, and the like, depending on the area affected. Of them, epithelial ovarian cancer accounts for approximately 90% of all ovarian cancer cases and is often found, particularly, in middle-aged and elderly persons over forties. Thus, the early detection of epithelial ovarian cancer, if possible, can decrease the mortality rate of ovarian cancer.

Unlike uterine cancer, neither can epithelial ovarian cancer be examined endoscopically nor its cells can be collected directly ab extra. Hence, laparotomy is required even for direct examination or cytological diagnosis. In addition, early detection by palpation is also difficult. The cancer is often undetected until its symptoms have progressed and the ovary has become enlarged. Although echography, MRI, CT, etc., is relatively effective for early detection, the examination itself is extensive work and entails high cost. Another problem of the examination is that the accuracy of benign or malignant diagnosis is not always high.

Against this background, tumor markers have received attention in recent years. The tumor markers refer to substances produced by cancer cells or substances produced by cells in response to cancer cells. The amounts of the tumor markers contained in body fluids such as serum reflect the amount, histological type, or grade (prognosis) of tumor. The tumor markers can therefore serve as an index for, for example, determining the presence or absence of cancer. The tumor markers have the advantages that for example, they permit examination using body fluids, leading to low invasiveness, and also enable such examination to be conducted conveniently with relatively low cost.

Various cancer-associated antigens such as CAl25, CA602, CA130, CA72-4, CA546, CA19-9, and STN have been known so far as tumor markers for epithelial ovarian cancer (Non Patent Literatures 1 to 6). All of these tumor markers, however, are based on the difference in expression level, i.e., increase or decrease in protein expression level, in serum between normal individuals and epithelial ovarian cancer patients. Such proteins are usually expressed in no small amount even in normal cells and are therefore low specific for epithelial ovarian cancer. Hence, these proteins produce high false-positive and false-negative rates and as such, are far from high accuracy as tumor markers. In addition, these tumor markers are used mainly for the diagnosis of prognosis of epithelial ovarian cancer. A tumor marker that contributes to the early detection of primary cancer still remains to be obtained.

### CITATION LIST

### Non Patent Literature

Non Patent Literature 1: Bast R.C. Jr. et al., 1983, N. Engl. J. Med., 309: 883-887
Non Patent Literature 2: Suzuki M. et al., 1990, Nippon Gan Chiryo Gakkai Shi, 25: 1454-1460
Non Patent Literature 3: Inaba N. et al., 1989, Nippon Gan Chiryo Gakkai Shi, 24: 2426-2435
Non Patent Literature 4: Ohuchi N. et al., 1988, Gan To Kagaku Ryoho, 15, 2767-2772
Non Patent Literature 5: Nozawa S. et al., 1996, Nippon Rinsho, 54: 1665-1673
Non Patent Literature 6: Charpin C. et al., 1982, Int. J. Gynecol. Pathol., 1: 231-245

### SUMMARY OF INVENTION

### Technical Problem

An object of the present invention is to develop and provide an epithelial ovarian cancer diagnosis biomarker with which epithelial ovarian cancer can be detected inexpensively, conveniently, and low invasively with high accuracy from a body fluid, a cell, or a peritoneal lavage fluid, and a method for determining the presence or absence of epithelial ovarian cancer using the marker.

### Solution to Problem

The composition, structures, and glycosylation sites of glycans that are linked to proteins secreted from cells are known to be controlled by the balanced expression of glycan-related genes and vary according to cell differentiation. The composition and structures of the glycans also vary according to the degree of cancer progression. Thus, glycoproteins found in particular cancer cells can be used as disease condition index markers including tumor markers. In recent years, such glycan-related tumor markers based on (glyco)proteomics have been searched for actively.

In order to attain the object, the present inventors have searched for epithelial ovarian cancer diagnosis markers using a lectin microarray method and glycoproteomics. As a result, the present inventors have successfully identified novel glycoprotein or glycopeptide groups having epithelial ovarian cancer-specific structures. The present inventors have also revealed that the presence or absence of epithelial ovarian cancer can be determined using these glycoprotein or glycopeptide groups. The present inventors have further found that the histological type of epithelial ovarian cancer can be determined by analogy using some members in these glycoprotein or glycopeptide groups. The present invention is based on these findings and provides the followings:

(1) A glycoprotein for an epithelial ovarian cancer diagnosis marker having at least one glycan-linked asparagine residue at a glycosylation site shown in Table 1 in the amino acid sequence of a protein shown in Table 1:

TABLE 1

| | | | | | Epithelial | | | |
|---|---|---|---|---|---|---|---|---|
| | | | | Glycosylation | ovarian cancer | | | SEQ |
| Protein # | Protein name | gi (ID) | | site | AAL(+) | WFA(+) | Peptide sequence | NO. |
| 1 | collagen, type VI, alpha 1 | gi\|87196339 | | 212 | ◯ | ◯ | RNFTAADWGQSR | 1 |
| 2 | biglycan | gi\|4502403 | | 270 | — | ◯ | MIENGSLSFLPTLR | 2 |
| | | | | 311 | ◯ | — | LLQVVYLHSNNITK | 3 |
| 3 | angiopoietin 2 | gi\|4557315 | | 240 | ◯ | ◯ | KIVTATVNNSVLQK | 4 |
| 4 | biotinidase | gi\|4557373 | | 349 | ◯ | ◯ | NPVGLIGAENATGETDPSHSK | 5 |
| 5 | follistatin-like 1 | gi\|5901956 | | 144 | ◯ | ◯ | RIIQWLEAEIIPDGWFSKGSNYSEILDK | 6 |
| 6 | L1 cell adhesion molecule 1 | gi\|4557707 | (isoform 1) | 671 | ◯ | — | WYSLGKVPGNQTSTTLK | 7 |
| | | | | 777 | ◯ | — | VQWRPQGTRGPWQEQIVSDPFLVVSNTSTFVPYEIK | 8 |
| | | | | 979 | — | ◯ | THNLTDLSPHLR | 9 |
| | | gi\|13435353 | (isoform 2) | 671 | ◯ | — | WYSLGKVPGNQTSTTLK | 7 |
| | | | | 777 | ◯ | — | VQWRPQGTRGPWQEQIVSDPFLVVSNTSTFVPYEIK | 8 |
| 7 | pregnancy specific beta-1-glycoprotein | gi\|21361296 | | 175 | ◯ | — | SENYTYIWWLNGQSLPVSPR | 10 |
| | 5 | | | 210 | — | ◯ | ILILPSVTRNETGPYECEIR | 11 |
| 8 | pregnancy specific beta-1-glycoprotein | gi\|21314635 | | 303 | ◯ | ◯ | ILILPSVTRNETGPYQCEIQDR | 12 |
| | 9 | | | | | | | |
| 9 | ribonuclease T2 | gi\|5231228 | | 106 | ◯ | — | AYWPDVIHSFPNRS | 13 |
| | | | | 212 | ◯ | ◯ | QDQQLQNCTEPGEQPSPK | 14 |
| 10 | tissue factor pathway inhibitor 2 | gi\|5730091 | | 116 | — | ◯ | YFFNLSSMTCEK | 15 |
| | | | | 170 | ◯ | ◯ | KIPSFCYSPKDEGLCSANVTR | 16 |
| 11 | tubulointerstitial nephritis antigen- | gi\|11545918 | | 78 | ◯ | ◯ | GRADDCALPYLGAICYCDLFCNRT | 17 |
| | like 1 | | | | | | | |
| 12 | cysteine-rich secretory protein 3 | gi\|5174675 | | 239 | ◯ | ◯ | DSCKASCNCSNSIY | 18 |
| 13 | histidine-rich glycoprotein | gi\|4504489 | | 125 | ◯ | ◯ | VIDFNCTTSSVSSALANTK | 19 |
| 14 | laminin, gamma 1 | gi\|145309326 | | 1161, 1175 | ◯ | — | AKVAAANVSVTQPESTGDPNNMTLLAEEAR | 20 |
| | | | | 1205 | ◯ | ◯ | TANDTSTEAYNLLLR | 21 |
| | | | | 1241 | ◯ | — | KYEQAKNISQDLEK | 22 |
| | | | | 1380 | ◯ | — | VNDNKTAAEEALRK | 23 |
| 15 | complement component 4 binding | gi\|4502503 | | 506, 528 | ◯ | ◯ | LSVDKDQYVEPENVTIQCDSGYGVVGPQSITCSGNRT | 24 |
| | protein, | | | | | | | |
| | alpha chain | | | | | | | |
| 16 | inter-alpha (globulin) inhibitor H4 | gi\|31542984 | | 517 | ◯ | ◯ | LPTQNITFQTESSVAEQEAEFQSPK | 25 |
| 17 | cathepsin L2 | gi\|23110960 | | 221 | ◯ | ◯ | YRPENSVANDTGFTVVAPGKEK | 26 |
| | | | | 292 | ◯ | ◯ | NLDHGVLVVGYGFEGANSNNSKYWLVK | 27 |
| 18 | collagen, type XII, alpha 1 | gi\|93141047 | (long isoform) | 2528 | ◯ | ◯ | MLEAYNLTEKNFASVQGVSLESGSFPSYSAYR | 28 |
| | | | | 2679 | ◯ | — | DIKEAGNITTDGYEILGK | 29 |
| | | gi\|93141049 | (short isoform) | 1364 | ◯ | — | MLEAYNLTEKNFASVQGVSLESGSFPSYSAYR | 28 |
| | | | | 1515 | ◯ | — | DIKEAGNITTDGYEILGK | 29 |
| 19 | kallikrein-related peptidase 5 | gi\|117306172 | | 173 | ◯ | ◯ | DVRPINVSSHCPSAGTK | 30 |
| | | gi\|117306176 | | 208 | ◯ | ◯ | VLQCLNISVLSQKR | 31 |
| | | gi\|6912644 | | | | | | |
| 20 | laminin alpha 3 subunit | gi\|38045910 | (isoform 1) | 2265 | ◯ | — | EVIDTNLTTLR | 32 |
| | | | | 2728 | ◯ | — | ERFNISTPAFR | 33 |
| | | | | 3097 | ◯ | — | EGSLPGNSTISIR | 34 |
| | | gi\|38045908 | (isoform 2) | 553 | — | ◯ | QLEEIKRNASGDELVR | 35 |
| | | | | 656 | ◯ | ◯ | EVIDTNLTTLR | 32 |
| | | | | 975 | — | ◯ | TFNLNTTEVEPCR | 36 |
| | | | | 1119 | ◯ | — | ERFNISTPAFR | 33 |
| | | | | 1488 | ◯ | ◯ | EGSLPGNSTISIR | 34 |
| 21 | netrin 4 | gi\|93204871 | | 56 | — | ◯ | KLWADTTCGQNATELYCFYSENTDLTCRQPK | 37 |
| | | | | 163 | ◯ | — | YFATNCSATFGLEDDVVKK | 38 |
| 22 | plasminogen activator inhibitor type | gi\|24307907 | | 118 | ◯ | ◯ | NKDIVTVANAVFVKNASEIEVPFVTR | 39 |
| | 1, | | | | | | | |
| | member 2 | | | | | | | |
| 23 | platelet derived growth factor D | gi\|13376808 | (isoform 1) | 276 | ◯ | ◯ | NYSVNIREELK | 40 |
| | | gi\|15451921 | (isoform 2) | 270 | ◯ | — | NYSVNIREELK | 40 |
| 24 | protease, serine, 23 | gi\|6005882 | | 93 | — | ◯ | QYLSYETLYANGSR | 41 |
| | | | | 207 | ◯ | ◯ | GANDSTSAMPEQMKFQWIR | 42 |
| 25 | semaphorin 5A | gi\|147904700 | | 147 | ◯ | ◯ | SLSNLTEIHDQISGMAR | 43 |
| | | | | 591 | ◯ | — | TRSCDSPAPQCGGWQCEGPGMEIANCSR | 44 |
| 26 | thrombospondin repeat containing 1 | gi\|38016904 | (isoform 1) | 773 | ◯ | — | CGHLPRPNITQSCQLR | 45 |
| | | | | 1001 | ◯ | ◯ | EVQCLSTNQTLSTR | 46 |
| | | gi\|56788359 | (isoform 2) | 773 | ◯ | — | CGHLPRPNITQSCQLR | 45 |
| 27 | complement component 1, | gi\|4502495 | | 174 | — | ◯ | NCGVNCSGDVFTALIGEIASPNYPKPYPENSR | 47 |
| | s subcomponent | gi\|41393602 | | 406 | ◯ | — | YTCEEPYYYMENGGGGEYHCAGNGSWVNEVLGPELPK | 48 |
| 28 | lysyl oxidase-like 2 | gi\|4505011 | | 288 | — | ◯ | LGPQVSLDPMKNVTCENGLPAVVSCVPGQVFSPDGPSR | 49 |
| | | | | 293 | — | ◯ | LGPQVSLDPMKNVTCENGLPAVVSCVPGQVFSPDGPSR | |
| | | | | 644 | ◯ | ◯ | HYHSMEVFTHYDLLNLNGTK | 50 |
| 29 | cathepsin D | gi\|4503143 | | 263 | ◯ | ◯ | YYKGSLSYLNVTR | 51 |
| 30 | fibrillin 1 | gi\|24430141 | | 448 | — | ◯ | VLPVNVTDYCQLVR | 52 |
| | | | | 1703, 1713 | ◯ | — | NYYADNQTCDGELLFNMTKK | 53 |
| 31 | peptidylprolyl isomerase B | gi\|4758950 | | 148 | ◯ | ◯ | HYGPGWVSMANAGKDTNGSQFFITTVK | 54 |
| 32 | attractin | gi\|21450861 | (isoform 1) | 264 | | ◯ | ISNSSDTVECECSENWK | 55 |
| | | | | 914, 923 | ◯ | ◯ | AATCINPLNGSVCERPANHSAK | 56 |
| | | | | 1073, 1082 | ◯ | — | CINQSICEKCENLTTGK | 57 |
| | | | | 1082 | ◯ | — | CENLTTGKHCETCISGFYGDPTNGGK | 58 |
| | | | | 1250, 1259 | — | ◯ | NHPNITFFVYVSNFTWPIK | 59 |
| | | gi\|21450863 | (isoform 2) | 914, 923 | ◯ | — | AATCINPLNGSVCERPANHSAK | 56 |
| | | | | 1073, 1082 | ◯ | — | CINQSICEKCENLTTGK | 57 |
| | | | | 1082 | ◯ | — | CENLTTGKHCETCISGFYGDPTNGGK | 58 |
| 33 | growth differentiation factor 15 | gi\|4758936 | | 70 | ◯ | ◯ | LRANQSWEDSNTDLVPAPAVR | 60 |
| 34 | heparan sulfate proteoglycan 2 | gi\|126012571 | | 3780 | — | ◯ | SLTQGSLIVGDLAPVNGTSQGK | 61 |
| | | | | 4068 | ◯ | — | SQGLNLHTLLYLGGVEPSVPLSPATNMSAHFR | 62 |
| 35 | ceruloplasmin | gi\|4557485 | | 138 | ◯ | ◯ | NLASRPYTFHSHGITYYKEHEGAIYPDNTTDFQR | 63 |
| | | | | 358 | ◯ | ◯ | AGLQAFFQVQECNKSSSK | 64 |
| | | | | 397 | ◯ | ◯ | ENLTAPGSDSAVFFEQGTTR | 65 |
| | | | | 762 | ◯ | ◯ | ELHHLQEQNVSNAFLDKGEFYIGSK | 66 |
| 36 | serine (or cysteine) proteinase | gi\|50363217 | | 70 | ◯ | — | QLAHQSNSTNIFFSPVSIATAFAMLSLGTK | 67 |
| | inhibitor, | gi\|50363219 | | 107 | ◯ | ◯ | ADTHDEILEGLNFNLTEIPEAQIHEGFQELLR | 68 |
| | clade A (alpha-1 antiproteinase, | gi\|50363221 | | 271 | ◯ | — | YLGNATAIFFLPDEGK | 69 |
| | antitrypsin), member 1 | | | | | | | |
| 37 | quiescin Q6 sulfhydryl oxidase 1 | gi\|13325075 | (isoform a) | 130 | ◯ | ◯ | AFTKNGSGAVFPVAGADVQTLR | 70 |
| | | gi\|51873067 | (isoform b) | 130 | ◯ | ◯ | AFTKNGSGAVFPVAGADVQTLR | 70 |
| 38 | golgi phosphoprotein 2 | gi\|29550838 | | 109 | ◯ | ◯ | LYQDEKAVLVNNITTGER | 71 |
| | | gi\|29550850 | | 144 | ◯ | ◯ | LQQDVLQFQKNQTNLER | 72 |
| 39 | galectin 3 binding protein | gi\|5031863 | | 69 | ◯ | ◯ | ALGFENATQALGR | 73 |
| | | | | 125 | — | ◯ | DAGVVCTNETR | 74 |
| | | | | 398 | ◯ | — | YKGLNLTEDTYKPR | 75 |
| | | | | 551 | ◯ | — | AAIPSALDTNSSK | 76 |
| | | | | 580 | ◯ | ◯ | TVIRPFYLTNSSGVD | 77 |
| 40 | tissue inhibitor of metalloproteinase | gi\|4507509 | | 53 | ◯ | ◯ | AKFVGTPEVNQTTLYQR | 78 |
| | 1 | | | 101 | ◯ | ◯ | SHNRSEEFLIAGK | 79 |
| 41 | agrin | gi\|54873613 | | 135 | ◯ | ◯ | NELMLNSSLMR | 80 |
| | | | | 250 | ◯ | — | DPCSNVTCSFGSTCAR | 81 |
| 42 | serpin peptidase inhibitor, clade G | gi\|73858568 | | 238 | ◯ | ◯ | DTFVNASR | 82 |
| | (C1 inhibitor), | gi\|73858570 | | 253 | ◯ | ◯ | VLSNNSDANLELINTWVAK | 83 |
| | member 1 | | | 352 | ◯ | ◯ | VGQLQLSHNLSLVILVPQNLK | 84 |
| 43 | complement factor H | gi\|62739186 | (isoform a) | 802 | ◯ | — | IPCSQPPQIEHGTINSSR | 85 |
| | | | | 802, 822 | ◯ | — | WDPEVNCSMAQIQLCPPPPQIPNSHNMTTTLNYR | 86 |
| | | | | 882 | ◯ | — | WQSIPLCVEKIPCSQPPQIEHGTINSSR | 87 |
| | | | | 911 | ◯ | ◯ | ISEENETTCYMGK | 88 |
| | | | | 1029 | ◯ | ◯ | MDGASNVTCINSR | 89 |
| | | | | 1095 | ◯ | — | YQCRSPYEMFGDEEVMCLNGNWTEPPQCK | 90 |
| 44 | kininogen 1 | gi\|4504893 | | 48 | — | ◯ | YNSQNQSNNQFVLYR | 91 |
| | | | | 169 | ◯ | — | HGIQYFNNNTQHSSLFMLNEVKR | 92 |
| | | | | 205 | ◯ | ◯ | ITYSIVQTNCSK | 93 |
| | | | | 294 | ◯ | ◯ | LNAENNATFYFK | 94 |
| 45 | vitronectin | gi\|88853069 | | 86 | ◯ | ◯ | NNATVHEQVGGPSLTSDLQAQSK | 95 |
| 46 | apolipoprotein D | gi\|4502163 | | 65 | — | ◯ | CIQANYSLMENGK | 96 |
| | | | | 98 | ◯ | ◯ | ADGTVNQIEGEATPVNLTEPAKLEVK | 97 |
| 47 | alpha-2-macroglobulin | gi\|66932947 | | 55, 70 | ◯ | — | GCVLLSYLNETVTVSASLESVRGNRS | 98 |
| | | | | 410, 413 | — | ◯ | GNEANYYSNATTDEHGLVQFSINTTNVMGTSLTVRVNYK | 99 |
| | | | | 1424 | ◯ | — | TEVSSNHVLIYLDKVSNQTLSLFFTVLQDVPVR | 100 |
| 48 | complement component 4B | gi\|50345296 | | 1328 | ◯ | ◯ | GLNVTLSSTGR | 101 |
| 49 | lumican | gi\|4505047 | | 88 | ◯ | — | NNQIDHIDEKAFENVTDLQWLILDHNLLENSK | 102 |
| | | | | 127 | ◯ | ◯ | KLHINHNNLTESVGPLPK | 103 |
| | | | | 160 | ◯ | ◯ | LGSFEGLVNLTFIHLQHNR | 104 |
| | | | | 252 | — | ◯ | LSHNELADSGIPGNSFNVSSLVELDLSYNK | 105 |
| 50 | phospholipid transfer protein | gi\|5453914 | (isoform a) | 64 | ◯ | — | GKEGHFYYNISEVK | 106 |
| | | | | 143 | ◯ | — | MKVSNVSCQASVSR | 107 |
| | | | | 398 | | ◯ | IYSNHSALESLALIPLQAPLK | 108 |
| | | gi\|33356541 | (isoform b) | 64 | ◯ | — | GKEGHFYYNISEVK | 106 |
| | | | | 346 | ◯ | — | IYSNHSALESLALIPLQAPLKTMLQIGVMPMLNER | 109 |
| 51 | prostaglandin H2 D-isomerase | gi\|32171249 | | 51 | ◯ | — | WFSAGLASNSSWLR | 110 |
| | | | | 78 | — | ◯ | SVVAPATDGGLNLTSTFLR | 111 |
| 52 | clusterin | gi\|42716297 | (isoform 1) | 138 | — | ◯ | EDALNETRESETKLK | 112 |
| | | | | 155 | ◯ | ◯ | LKELPGVCNETMMALWEECKPCLK | 113 |
| | | | | 406 | ◯ | ◯ | MLNTSSLLEQLNEQFNWVSR | 114 |
| | | | | 406, 426 | ◯ | ◯ | MLNTSSLLEQLNEQFNWVSRLANLTQGEDQYYLR | 115 |
| | | | | 426 | ◯ | ◯ | LANLTQGEDQYYLR | 116 |
| | | gi\|42740907 | (isoform 2) | 103 | ◯ | — | LKELPGVCNETMMALWEECKPCLK | 113 |
| | | | | 354 | ◯ | — | MLNTSSLLEQLNEQFNWVSR | 114 |
| | | | | 354, 374 | ◯ | — | MLNTSSLLEQLNEQFNWVSRLANLTQGEDQYYLR | 115 |
| | | | | 374 | ◯ | — | LANLTQGEDQYYLR | 116 |
| 53 | complement factor B | gi\|67782358 | | 122 | ◯ | ◯ | SPYYNVSDEISFHCYDGYTLR | 117 |
| 54 | alpha-2-HS-glycoprotein | gi\|4502005 | | 156 | ◯ | ◯ | KVCQDCPLLAPLNDTR | 118 |
| | | | | 176 | ◯ | ◯ | AALAAFNAQNNGSNFQLEEISR | 119 |
| 55 | polymeric immunoglobulin receptor | gi\|31377806 | | 83, 90 | ◯ | — | ANLTNFPENGTFVVNIAQLSQDDSGRYK | 120 |
| | | | | 186 | ◯ | — | QIGLYPVLVIDSSGYVNPNYTGR | 121 |
| | | | | 421 | — | ◯ | LSLLEEPGNGTFTVILNQLTSR | 122 |
| | | | | 469 | — | ◯ | IIEGEPNLKVPGNVTAVLGETLK | 123 |
| 56 | hemopexin | gi\|11321561 | | 187 | ◯ | ◯ | SWPAVGNCSSALR | 124 |
| | | | | 240, 246 | ◯ | ◯ | GHGHRNGTGHGNSTHHGPEYMR | 125 |
| | | | | 453 | ◯ | ◯ | ALPQPQNVTSLLGCTH | 126 |
| 57 | orosomucoid 1 | gi\|9257232 | | 56 | ◯ | — | WFYIASAFRNEEYNKS | 127 |
| | | | | 72 | ◯ | ◯ | SVQEIQATFFYFTPNKTEDTIFLR | 128 |
| | | | | 93 | ◯ | — | QDQCIYNTTYLNVQR | 129 |
| | | | | 93, 98 | — | ◯ | EYQTRQDQCIYNTTYLNVQRENGTISR | 130 |
| | | | | 93, 103 | ◯ | — | QDQCIYNTTYLNVQRENGTISR | 131 |
| 58 | orosomucoid 2 | gi\|4505529 | | 56 | ◯ | — | WFYIASAFRNEEYNKS | 127 |
| | | | | 72 | ◯ | — | SVQEIQATFFYFTPNKTEDTIFLR | 128 |
| | | | | 93 | ◯ | — | QNQCFYNSSYLNVQR | 132 |
| | | | | 93, 103 | ◯ | ◯ | QNQCFYNSSYLNVQRENGTVSR | 133 |
| 59 | serpin peptidase inhibitor, | gi\|50659080 | | 106 | ◯ | — | FNLTETSEAEIHQSFQHLLR | 134 |
| | clade A, member 3 | | | 127 | — | ◯ | TLNQSSDELQLSMGNAMFVK | 135 |
| | | | | 186 | ◯ | — | LINDYVKNGTR | 136 |
| | | | | 271 | ◯ | ◯ | YTGASALFILPDQDKMEEVEAMLLPETLK | 137 |
| 60 | transferrin | gi\|4557871 | | 430 | — | ◯ | CGLVPVLAENYNK | 138 |
| | | | | 432 | ◯ | — | CGLVPVLAENYNKSDNCEDTPEAGYFAVAVVK | 139 |
| | | | | 630 | ◯ | ◯ | QQQHLFGSNVTDCSGNFCLFRSETK | 140 |
| 61 | alpha-2-glycoprotein 1, zinc | gi\|4502337 | | 109, 112 | ◯ | — | DIVEYYNDSNGSHVLQGR | 141 |
| | | | | 112 | ◯ | ◯ | DIVEYYNDSNGSHVLQGR | |
| | | | | 128 | — | ◯ | FGCEIENNRSSGAFWK | 142 |
| 62 | laminin alpha 5 | gi\|21264602 | | 95 | — | ◯ | LVGGPVAGGDPNQTIR | 143 |
| | | | | 921 | ◯ | — | LNLTSPDLFWLVFR | 144 |
| | | | | 1330 | ◯ | — | VWQGHANASFCPHGYGCR | 145 |
| | | | | 2196 | ◯ | — | GINASSMAWAR | 146 |
| | | | | 2209 | ◯ | — | LHRLNASIADLQSQLR | 147 |
| | | | | 2707 | ◯ | — | GVHNASLALSASIGR | 148 |
| 63 | lunatic fringe | gi\|93140999 | (isoform a) | 167 | ◯ | ◯ | HTGNVVITNCSAAHSR | 149 |
| | | gi\|93141005 | (isoform b) | 167 | ◯ | ◯ | HTGNVVITNCSAAHSR | 149 |
| 64 | met proto-oncogene | gi\|42741655 | | 399 | ◯ | — | CLQHFYGPNHEHCFNRT | 150 |
| | | | | 607 | — | ◯ | VLLGNESCTLTLSESTMNTLK | 151 |
| | | | | 785 | ◯ | — | MVINVHEAGRNFTVACQHR | 152 |
| 65 | prion protein | gi\|122056623 | | 197 | ◯ | ◯ | QHTVTTTTKGENFTETDVK | 153 |
| | | gi\|122056625 | | | | | | |
| | | gi\|122056628 | | | | | | |
| | | gi\|34335270 | | | | | | |
| | | gi\|4506113 | | | | | | |
| 66 | insulin-like growth factor binding | gi\|62243248 | (isoform a) | 116 | ◯ | — | GLCVNASAVSR | 154 |
| | protein 3 | gi\|62243068 | (isoform b) | 116 | ◯ | ◯ | GLCVNASAVSR | 154 |
| | | | | 136 | — | ◯ | LRAYLLPAPPAPGNASESEEDRSAGSVESPSVSSTHR | 155 |
| | | | | 199 | — | ◯ | YKVDYESQSTDTQNFSSESKR | 156 |
| 67 | lipocalin 2 | gi\|38455402 | | 85 | ◯ | ◯ | MYATIYELKEDKSYNVTSVLFR | 157 |
| 68 | coagulation factor XII | gi\|145275213 | | 249 | ◯ | — | TTLSGAPCQPWASEATYRNVTAEQAR | 158 |
| 69 | decorin | gi\|19743846 | (isoform a) | 262 | ◯ | ◯ | LGLSFNSISAVDNGSLANTPHLR | 159 |
| | | gi\|4503271 | | | | | | |
| | | gi\|19743848 | (isoform b) | 153 | ◯ | — | LGLSFNSISAVDNGSLANTPHLR | 159 |
| | | gi\|19743850 | (isoform c) | 115 | ◯ | — | LGLSFNSISAVDNGSLANTPHLR | 159 |
| 70 | amyloid beta A4 protein | gi\|4502167 | (isoform a) | 571 | ◯ | — | EQNYSDDVLANMISEPR | 160 |
| | | gi\|41406055 | (isoform b) | 552 | ◯ | — | EQNYSDDVLANMISEPR | 160 |
| | | gi\|41406057 | (isoform c) | 496 | ◯ | — | EQNYSDDVLANMISEPR | 160 |
| 71 | chorionic gonadotropin | gi\|4502789 | (beta 3 subunit) | 33 | ◯ | — | CRPINATLAVEK | 161 |
| | | gi\|132566537 | (beta polypeptide 1) | 31 | ◯ | — | CRPINATLAVEK | 161 |
| | | gi\|132566538 | (beta polypeptide 2) | 31 | ◯ | — | CRPINATLAVEK | 161 |
| | | gi\|15451748 | (beta polypeptide 5) | 33 | ◯ | — | CRPINATLAVEK | 161 |
| | | gi\|15451750 | (beta polypeptide 7) | 33 | ◯ | — | CRPINATLAVEK | 161 |
| | | gi\|15426252 | (beta polypeptide 8) | 33 | ◯ | — | CRPINATLAVEK | 161 |
| 72 | colony stimulating factor 1 | gi\|27262661 | (isoform a) | 154 | ◯ | — | NVFNETKNLLDKDWNIFSK | 162 |
| | | gi\|27262667 | | | | | | |
| | | gi\|27262663 | (isoform b) | 154 | ◯ | — | NVFNETKNLLDKDWNIFSK | 162 |
| | | gi\|27262665 | (isoform c) | 154 | ◯ | — | NVFNETKNLLDKDWNIFSK | 162 |
| 73 | cystatin M | gi\|4503113 | | 137 | ◯ | — | LRCDFEVLVVPWQNSSQLLK | 163 |
| 74 | endothelin converting enzyme 1 | gi\|4503443 | | 166 | ◯ | — | HLLENSTASVSEAERK | 164 |
| 75 | FK506 binding protein 10, 65 kDa | gi\|21361895 | | 70 | ◯ | — | YHYNGTFEDGKK | 165 |
| 76 | fucosidase, alpha-L-1, tissue | gi\|119360348 | | 268 | ◯ | — | WGQNCSCHHGGYYNCEDK | 166 |
| 77 | heparan sulfate 6-0-sulfotransferase 1 | gi\|148747866 | | 320 | ◯ | — | FIRPFMQYNSTR | 167 |
| 78 | insulin-like growth factor binding | gi\|4504619 | | 171 | ◯ | — | GTCEQGPSIVTPPKDIWNVTGAQVYLSCEVIGIPTPVLIWNK | 168 |
| | protein 7 | | | | | | | |
| 79 | lectin, mannose-binding 2 | gi\|5803023 | | 183 | ◯ | — | VFPYISVMVNNGSLSYDHSKDGR | 169 |
| 80 | leukemia inhibitory factor | gi\|4504991 | | 85, 95 | ◯ | — | LCGPNVTDFPPFHANGTEK | 170 |
| | (cholinergic differentiation factor) | | | | | | | |
| 81 | notch 2 | gi\|24041035 | | 46 | ◯ | — | DGYEPCVNEGMCVTYHNGTGYCK | 171 |
| 82 | osteoprotegerin | gi\|148743793 | | 289 | ◯ | — | HIGHANLTFEQLR | 172 |
| 83 | poliovirus receptor | gi\|19923372 | | 120 | ◯ | — | VEDEGNYTCLFVTFPQGSR | 173 |
| 84 | pregnancy specific beta-1-glycoprotein | gi\|21361392 | | 259, 268 | ◯ | — | ENKDVLNFTCEPKSENYTYIWWLNGQSLPVSPR | 174 |
| | 1 | | | 268 | ◯ | — | SENYTYIWWLNGQSLPVSPR | 10 |
| 85 | pregnancy specific beta-1-glycoprotein | gi\|109240546 | | 268 | ◯ | — | SENYTYIWWLNGQSLPVSPR | 10 |
| | 3 | | | 303 | ◯ | — | ILILPSVTRNETGPYQCEIQDR | 12 |
| 86 | pregnancy specific beta-1-glycoprotein | gi\|42560235 | | 299, 303 | ◯ | — | ILILPNVTRNETGPYQCEIR | 175 |
| | 4 | | | | | | | |
| 87 | pregnancy specific beta-1-glycoprotein | gi\|4506177 | | 268 | ◯ | — | SENYTYIWWLNGQSLPVSPR | 10 |
| | 7 | | | | | | | |
| 88 | pregnancy specific beta-1-glycoprotein | gi\|51510887 | | 259, 268 | ◯ | — | ENKDVLNFTCEPKSENYTYIWWLNGQSLPVSPR | 174 |
| | 8 | | | 268 | ◯ | — | SENYTYIWWLNGQSLPVSPR | 10 |
| 89 | solute carrier family 39 (zinc | gi\|55741750 | | 191, 198 | ◯ | — | LHHHLDHNNTHHFHNDSITPSER | 176 |
| | transporter), | | | | | | | |
| | member 10 | | | | | | | |
| 90 | twisted gastrulation | gi\|10190664 | | 52 | ◯ | — | CLIQELCQCRPGEGNCSCCK | 177 |
| 91 | TYR03 protein tyrosine kinase | gi\|27597078 | | 240 | ◯ | — | LSSSNASVAWMPGADGR | 178 |
| 92 | glutaminyl-peptide cyclotransferase | gi\|6912618 | | 296 | ◯ | — | YFQNYSYGGVIQDDHIPFLRR | 179 |
| 93 | alpha 1,4-galactosyltransferase | gi\|8392830 | | 121 | ◯ | — | GLPGGNASLPR | 180 |
| 94 | glucocerebrosidase | gi\|54607043 | | 309 | ◯ | — | DLGPTLANSTHHNVR | 181 |
| | | gi\|54607045 | | | | | | |
| | | gi\|54607047 | | | | | | |
| | | gi\|54607049 | | | | | | |
| | | gi\|54607051 | | | | | | |
| 95 | lysosomal-associated membrane protein | gi\|4504957 | | 257 | ◯ | — | VASVININPNTTHSTGSCR | 182 |
| | 2 | gi\|7669503 | | | | | | |
| 96 | quiescin Q6-like 1 | gi\|145580631 | | 266 | ◯ | — | LGVSSVPSCYLIYPNGSHGLINVVKPLR | 183 |
| 97 | transmembrane emp24 protein | gi\|33457308 | | 117 | ◯ | — | FTFTSHTPGDHQICLHSNSTR | 184 |
| | transport domain containing 4 | | | | | | | |
| 98 | neuropilin 1 | gi\|66912184 | (isoform a) | 522 | ◯ | — | FKIGYSNNGSDWK | 185 |
| | | gi\|66912178 | (isoform b) | 522 | ◯ | — | FKIGYSNNGSDWK | 185 |
| | | gi\|66864913 | (isoform c) | 522 | ◯ | — | FKIGYSNNGSDWK | 185 |
| 99 | versican | gi\|21361116 | | 1442 | ◯ | — | RGQFESVAPSQNFSDSSESDTHPFVIAK | 186 |
| 100 | coagulation factor II | gi\|4503635 | | 416 | ◯ | — | WVLTAAHCLLYPPWDKNFTENDLLVR | 187 |
| 101 | fibronectin 1 | gi\|47132557 | (isoform 1) | 542 | ◯ | — | RHEEGHMLNCTCFGQGR | 188 |
| | | gi\|47132551 | (isoform 2) | 542 | ◯ | — | RHEEGHMLNCTCFGQGR | 188 |
| | | gi\|16933542 | (isoform 3) | 542 | ◯ | — | RHEEGHMLNCTCFGQGR | 188 |
| | | gi\|47132555 | (isoform 4) | 542 | ◯ | — | RHEEGHMLNCTCFGQGR | 188 |
| | | gi\|47132553 | (isoform 5) | 542 | ◯ | — | RHEEGHMLNCTCFGQGR | 188 |
| | | gi\|47132549 | (isoform 6) | 542 | ◯ | — | RHEEGHMLNCTCFGQGR | 188 |
| | | gi\|47132547 | (isoform 7) | 542 | ◯ | — | RHEEGHMLNCTCFGQGR | 188 |
| 102 | folate receptor 1 | gi\|4758400 | | 161 | ◯ | — | GWNWTSGFNK | 189 |
| | | gi\|9257205 | | | | | | |
| | | gi\|9257207 | | | | | | |
| | | gi\|9257213 | | | | | | |
| | | gi\|9257215 | | | | | | |
| | | gi\|9257217 | | | | | | |
| 103 | immunoglobulin superfamily, member 1 | gi\|45505167 | | 1223 | ◯ | — | GIGNYSCSYR | 190 |
| 104 | lactotransferrin | gi\|54607120 | | 156 | ◯ | — | TAGWNVPIGTLRPFLNWTGPPEPIEAAVAR | 191 |
| 105 | plasma carboxypeptidase B2 | gi\|126273569 | (isoform a) | 85, 108 | ◯ | — | AHLNVSGIPCSVLLADVEDLIQQQISNDTVSPR | 192 |
| | | gi\|126273559 | (isoform b) | 85, 108 | ◯ | — | AHLNVSGIPCSVLLADVEDLIQQQISNDTVSPR | 192 |
| 106 | plasma kallikrein B1 | gi\|78191798 | | 396 | ◯ | — | IVGGTNSSWGEWPWQVSLQVK | 193 |
| 107 | secreted frizzled-related protein 4 | gi\|4506895 | | 38 | ◯ | — | HMPWNITR | 194 |
| 108 | CD163 antigen | gi\|44662834 | (isoform a) | 140 | ◯ | — | HSNCTHQQDAGVTCSDGSNLEMR | 195 |
| | | gi\|44889963 | (isoform b) | 140 | ◯ | — | HSNCTHQQDAGVTCSDGSNLEMR | 195 |
| 109 | complement component 3 | gi\|115298678 | | 85 | ◯ | — | TVLTPATNHMGNVTFTIPANR | 196 |
| 110 | mucin 16 | gi\|83367077 | | 12586 | ◯ | — | NTSVGLLYSGCR | 197 |
| | | | | 13193 | ◯ | — | KFNITESVLQGLLKPLFK | 198 |
| | | | | 14417, 14423 | ◯ | — | NGTQLQNFTLDR | 199 |
| 111 | acid alpha-glucosidase | gi\|119393891 | | 470 | ◯ | — | GVFITNETGQPLIGK | 200 |
| | | gi\|119393893 | | | | | | |
| | | gi\|119393895 | | | | | | |
| 112 | activating transcription factor 6 | gi\|56786157 | | 584 | ◯ | — | DHLLLPATTHNKTTRPK | 201 |
| 113 | bone morphogenetic protein 1 | gi\|4502421 | (isoform 1) | 599 | ◯ | — | LNGSITSPGWPK | 202 |
| | | gi\|5902808 | (isoform 2) | 599 | ◯ | — | LNGSITSPGWPK | 202 |
| | | gi\|5453579 | (isoform 3) | 599 | ◯ | — | LNGSITSPGWPK | 202 |
| 114 | cAMP responsive element binding | gi\|20631977 | | 610 | ◯ | — | DHLLLPAISHNKTSRPK | 203 |
| | protein-like 1 | | | | | | | |
| 115 | carboxylesterase 1 | gi\|68508967 | (isoform a) | 80 | ◯ | — | NATSYPPMCTQDPK | 204 |
| | | gi\|68508965 | (isoform b) | 79 | ◯ | — | NATSYPPMCTQDPK | 204 |
| | | gi\|68508957 | (isoform c) | 79 | ◯ | — | NATSYPPMCTQDPK | 204 |
| 116 | carboxypeptidase D | gi\|22202611 | | 522 | ◯ | — | RFANEYPNITR | 205 |
| | | | | 978 | ◯ | — | HIWSLEISNKPNVSEPEEPKIR | 206 |
| | | | | 1070 | ◯ | — | GKDLDTDFTNNASQPETK | 207 |
| 117 | carboxypeptidase E | gi\|4503009 | | 139 | ◯ | — | ELLIFLAQYLCNEYQKGNETIVNLIHSTR | 208 |
| 118 | cathepsin F | gi\|6042196 | | 195 | ◯ | — | NFVITYNRT | 209 |
| 119 | cathepsin L1 | gi\|22202619 | | 221 | ◯ | — | YSVANDTGFVDIPKQEK | 210 |
| | | gi\|4503155 | | | | | | |
| 120 | CD276 antigen | gi\|67188443 | (isoform a) | 91, 104 | ◯ | — | QLVHSFAEGQDQGSAYANRTALFPDLLAQGNASLR | 211 |
| | | | | 215 | ◯ | — | VVLGANGTYSCLVR | 212 |
| | | | | 309, 332 | ◯ | — | DQGSAYANRTALFPDLLAQGNASLR | 213 |
| | | gi\|13376852 | (isoform b) | 91, 104 | ◯ | — | QLVHSFAEGQDQGSAYANRTALFPDLLAQGNASLR | 211 |
| | | | | 215 | ◯ | — | VVLGANGTYSCLVR | 212 |
| 121 | chitobiase, di-N-acetyl- | gi\|4758092 | | 299 | ◯ | — | QINSSISGNLWDK | 214 |
| 122 | chromosome 3 open reading frame 9 | gi\|23308761 | | 373 | ◯ | — | FLSYNVTR | 215 |
| | | gi\|31982953 | | | | | | |
| 123 | coagulation factor III | gi\|4503641 | | 169 | ◯ | — | NNTFLSLR | 216 |
| 124 | epidermal growth factor receptor | gi\|29725609 | (isoform a) | 603 | ◯ | — | TCPAGVMGENNTLVWK | 217 |
| | | gi\|41327732 | (isoform b) | 603 | ◯ | — | TCPAGVMGENNTLVWK | 217 |
| | | gi\|41327736 | (isoform d) | 603 | ◯ | — | TCPAGVMGENNTLVWK | 217 |
| 125 | GalNAc transferase 10 | gi\|38195091 | (isoform a) | 146 | ◯ | — | YLETLPNTSIIIPFHNEGWSSLLR | 218 |
| 126 | heparan sulfate 6-0-sulfotransferase 3 | gi\|45580707 | | 380 | ◯ | — | FISPFTQFNITR | 219 |
| 127 | heparan sulfate D-glucosaminyl | gi\|4826764 | | 242, 249 | ◯ | — | LSPQINASNFYFNKT | 220 |
| | 3-0-sulfotransferase 1 | | | | | | | |
| 128 | granulin | gi\|4504151 | | 236 | ◯ | — | YGCCPMPNATCCSDHLHCCPQDTVCDLIQSK | 221 |
| 129 | integrin alpha 3 | gi\|4504747 | (isoform a) | 969 | ◯ | — | TSIPTINMENKT | 222 |
| | | gi\|6006011 | (isoform b) | 969 | ◯ | — | TSIPTINMENKT | 222 |
| 130 | integrin alpha-V | gi\|4504763 | | 74 | ◯ | — | MFLLVGAPKANTTQPGIVEGGQVLK | 223 |
| | | | | 704 | ◯ | — | LSCAFKTENQTR | 224 |
| 131 | laminin, beta 2 | gi\|119703755 | | 1308 | ◯ | — | LALNLTLR | 225 |
| | | | | 1348 | ◯ | — | RANTSALAVPSPVSNSASAR | 226 |
| 132 | laminin subunit beta 3 | gi\|62868215 | | 604 | ◯ | — | LRNATASLWSGPGLEDR | 227 |
| | | gi\|62868217 | | | | | | |
| 133 | low density lipoprotein-related | gi\|126012562 | | 1511 | ◯ | — | ANKWTGHNVTVVQR | 228 |
| | protein 1 | | | | | | | |
| 134 | mannosidase, alpha, class 2A, member 1 | gi\|51477714 | | 1125 | ◯ | — | LLNKFIVESLTPSSLSLMHSPPGTQNISEINLSPMEISTFR | 229 |
| 135 | mannosidase, alpha, class 2B, member 2 | gi\|50659093 | | 748 | ◯ | — | RPYVSYVNNSIAR | 230 |
| 136 | mannosidase, beta A, lysosomal | gi\|84798622 | | 77 | ◯ | — | WVSLDNWTYSK | 231 |
| 137 | multiple EGF-like-domains 8 | gi\|145701025 | | 1204 | ◯ | — | ALLTNVSSVALGSR | 232 |
| | | | | 2147, 2162 | ◯ | — | TGYTMDNMTGLCRPVCAQGCVNGSCVEPDHCR | 233 |
| 138 | oxidized low density lipoprotein | gi\|4505501 | | 183 | ◯ | — | LLKINSTADLDFIQQAISYSSFPFWMGLSR | 234 |
| | (lectin-like) receptor 1 | | | | | | | |
| 139 | peptidylprolyl isomerase A | gi\|10863927 | | 108 | ◯ | — | HTGPGILSMANAGPNTNGSQFFICTAK | 235 |
| 140 | plexin A1 | gi\|49355818 | | 54 | ◯ | — | LSGNLTLLR | 236 |
| 141 | prominin 2 | gi\|21389623 | | 725 | ◯ | — | ILRNVSECFLAR | 237 |
| 142 | protein tyrosine phosphatase, | gi\|148728162 | (isoform 1) | 413 | ◯ | — | IHVAGETDSSNLNVSEPR | 238 |
| | receptor type, J | gi\|148728160 | (isoform 2) | 413 | ◯ | — | IHVAGETDSSNLNVSEPR | 238 |
| 143 | procollagen-lysine, | gi\|33636742 | (isoform a) | 717 | ◯ | — | FLRYNCSIESPR | 239 |
| | 2-oxoglutarate 5-dioxygenase 2 | gi\|62739166 | (isoform b) | 696 | ◯ | — | FLRYNCSIESPR | 239 |
| 144 | protein tyrosine phosphatase, receptor | gi\|18860902 | | 416 | ◯ | — | RIAVDWESLGYNITR | 240 |
| | type, K | | | 462 | ◯ | — | APQHVVNHLPPYTNVSLK | 241 |
| 145 | proton-coupled folate transporter | gi\|31543204 | | 58 | ◯ | — | FSADLGYNGTR | 242 |
| 146 | semaphorin 3F | gi\|31377802 | | 126 | ◯ | — | LIQPWNRT | 243 |
| 147 | sialyltransferase 4A | gi\|27765096 | | 323 | ◯ | — | TGVHDADFESNVTATLASINK | 244 |
| | | gi\|4506951 | | | | | | |
| 148 | solute carrier family 44, member 2 | gi\|31377727 | | 200 | ◯ | — | KNITDLVEGAK | 245 |
| 149 | tetraspan NET-6 | gi\|7657373 | | 137 | ◯ | — | SVNPNDTCLASCVK | 246 |
| 150 | transmembrane 4 superfamily member 15 | gi\|6912530 | | 189 | ◯ | — | NTTEVVNTMCGYK | 247 |
| 151 | transmembrane protein 132A | gi\|30089935 | (isoform a) | 280 | ◯ | — | HNFTASLLTLR | 248 |
| | | gi\|30089937 | (isoform b) | 280 | ◯ | — | HNFTASLLTLR | 248 |
| 152 | tumor necrosis factor receptor | gi\|7657039 | | 278 | ◯ | — | VLSSIQEGTVPDNTSSAR | 249 |
| | superfamily, | | | | | | | |
| | member 21 | | | | | | | |
| 153 | complement component 7 | gi\|45580688 | | 754 | ◯ | — | NYTLTGRDSCTLPASAEK | 250 |
| 154 | WAP four-disulfide core domain 2 | gi\|56699495 | | 44 | ◯ | — | TGVCPELQADQNCTQECVSDSECADNLK | 251 |
| 155 | CD82 antigen | gi\|4504813 | (isoform 1) | 129 | ◯ | — | DYNSSREDSLQDAWDYVQAQVK | 252 |
| | | gi\|67782354 | (isoform 2) | 104 | ◯ | — | DYNSSREDSLQDAWDYVQAQVK | 252 |
| 156 | FAT tumor suppressor 1 | gi\|66346693 | | 998 | ◯ | — | QVYNLTVR | 253 |
| 157 | membrane-bound transcription factor | gi\|4506775 | | 148 | ◯ | — | YAESDPTVPCNETR | 254 |
| | site-1 protease | | | 939 | ◯ | — | LSWAKPQPLNETAPSNLWK | 255 |
| 158 | poliovirus receptor-related 1 | gi\|42560237 | (isoform 1) | 286 | ◯ | — | ADANPPATEYHWTTLNGSLPK | 256 |
| | (herpesvirus entry mediator C; nectin) | gi\|42560231 | (isoform 2) | 286 | ◯ | — | ADANPPATEYHWTTLNGSLPK | 256 |
| | | gi\|42560233 | (isoform 3) | 286 | ◯ | — | ADANPPATEYHWTTLNGSLPK | 256 |
| 159 | solute carrier family 3 | gi\|61744475 | (isoform a) | 396 | ◯ | — | DIENLKDASSFLAEWQNITK | 257 |
| | (activators of dibasic and neutral | | | 455 | ◯ | — | SLVTQYLNATGNRWCSWSLSQAR | 258 |
| | amino acid transport), | gi\|61744477 | (isoform b) | 366 | ◯ | — | DIENLKDASSFLAEWQNITK | 257 |
| | member 2 | | | 425 | ◯ | — | SLVTQYLNATGNRWCSWSLSQAR | 258 |
| | | gi\|65506891 | (isoform c) | 365 | ◯ | — | DIENLKDASSFLAEWQNITK | 257 |
| | | | | 424 | ◯ | — | SLVTQYLNATGNRWCSWSLSQAR | 258 |
| | | gi\|61744479 | (isoform d) | 334 | ◯ | — | DIENLKDASSFLAEWQNITK | 257 |
| | | | | 393 | ◯ | — | SLVTQYLNATGNRWCSWSLSQAR | 258 |
| | | gi\|61744481 | (isoform e) | 303 | ◯ | — | DIENLKDASSFLAEWQNITK | 257 |
| | | | | 362 | ◯ | — | SLVTQYLNATGNRWCSWSLSQAR | 258 |
| | | gi\|61744483 | (isoform f) | 264 | ◯ | — | DIENLKDASSFLAEWQNITK | 257 |
| | | | | 323 | ◯ | — | SLVTQYLNATGNRWCSWSLSQAR | 258 |
| 160 | activated leukocyte cell adhesion | gi\|68163411 | | 167 | ◯ | — | KLGDCISEDSYPDGNITWYR | 259 |
| | molecule | | | 265 | | | NAIKEGDNITLK | 260 |
| | | | | 361 | | | NATVVWMKDNIR | 261 |
| | | | | 480 | | | IIISPEENVTLTCTAENQLER | 262 |
| 161 | F11 receptor | gi\|21464111 | | 185 | ◯ | — | AFSNSSYVLNPTTGELVFDPLSASDTGEYSCEAR | 263 |
| | | gi\|8393638 | | | | | | |
| 162 | basigin | gi\|38372919 | (isoform 1) | 160 | ◯ | — | ILLTCSLNDSATEVTGHR | 264 |
| | | | | 268 | ◯ | — | ITDSEDKALMNGSESR | 265 |
| | | gi\|38372925 | (isoform 2) | 44 | ◯ | — | ILLTCSLNDSATEVTGHR | 264 |
| | | | | 152 | ◯ | — | ITDSEDKALMNGSESR | 265 |
| | | gi\|38372921 | (isoform 3) | 59 | ◯ | — | ITDSEDKALMNGSESR | 265 |
| | | gi\|38372923 | (isoform 4) | 88 | ◯ | — | ITDSEDKALMNGSESR | 265 |
| 163 | CD63 antigen | gi\|4502679 | (isoform A) | 130 | ◯ | — | QQMENYPKNNHTASILDR | 266 |
| | | | | 150 | ◯ | — | CCGAANYTDWEKIPSMSK | 267 |
| | | gi\|91199546 | (isoform B) | 130 | ◯ | — | QQMENYPKNNHTASILDR | 266 |
| | | | | 150 | ◯ | — | CCGAANYTDWEKIPSMSK | 267 |
| 164 | integrin beta 1 | gi\|19743813 | (isoform 1A) | 212 | ◯ | — | LRNPCTSEQNCTSPFSYK | 268 |
| | | gi\|19743823 | | | | | | |
| | | gi\|19743815 | (isoform 1B) | 212 | ◯ | — | LRNPCTSEQNCTSPFSYK | 268 |
| | | gi\|19743817 | (isoform 1C-1) | 212 | ◯ | — | LRNPCTSEQNCTSPFSYK | 268 |
| | | gi\|19743821 | (isoform 1C-2) | 212 | ◯ | — | LRNPCTSEQNCTSPFSYK | 268 |
| | | gi\|19743819 | (isoform 1D) | 212 | ◯ | — | LRNPCTSEQNCTSPFSYK | 268 |
| 165 | Na+/K+ -ATPase beta 1 subunit | gi\|4502277 | (isoform a) | 158 | ◯ | — | FKLEWLGNCSGLNDETYGYK | 269 |
| | | | | 193 | ◯ | — | VLGFKPKPPKNESLETYPVMK | 270 |
| | | gi\|49574489 | (isoform b) | 158 | ◯ | — | FKLEWLGNCSGLNDETYGYK | 269 |
| | | | | 193 | ◯ | — | VLGFKPKPPKNESLETYPVMK | 270 |
| 166 | scavenger receptor class B, member 2 | gi\|5031631 | | 105 | ◯ | — | ANIQFGDNGTTISAVSNK | 271 |
| 167 | tumor-associated calcium signal | gi\|4505057 | | 168 | ◯ | — | HRPTAGAFNHSDLDAELRR | 272 |
| | transducer 2 | | | | | | | |
| 168 | carcinoembryonic antigen-related | gi\|98986445 | | 204 | ◯ | — | TLTLFNVTR | 273 |
| | cell adhesion molecule 5 | | | | | | | |
| 169 | cathepsin H | gi\|23110955 | (isoform a) | 101 | ◯ | — | HKYLWSEPQNCSATK | 274 |
| | | gi\|23110957 | (isoform b) | 89 | ◯ | — | HKYLWSEPQNCSATK | 274 |
| 170 | extracellular matrix protein 1 | gi\|4758236 | (isoform 1) | 444 | ◯ | — | HIPGLIHNMTAR | 275 |
| | | gi\|12707572 | (isoform 2) | 319 | ◯ | — | HIPGLIHNMTAR | 275 |
| 171 | complement factor I | gi\|119392081 | | 103 | — | ◯ | FLNNGTCTAEGK | 276 |
| | | | | 177 | ◯ | — | FKLSDLSINSTECLHVHCR | 277 |
| | | | | 464 | ◯ | — | SIPACVPWSPYLFQPNDTCIVSGWGR | 278 |
| 172 | multimerin 2 | gi\|13376091 | | 845 | ◯ | — | FNTTYINIGSSYFPEHGYFR | 279 |
| 173 | plexin domain containing 2 | gi\|40255005 | | 160 | ◯ | — | VNLSFDFPFYGHFLR | 280 |
| 174 | selectin L | gi\|4506875 | | 60 | ◯ | — | FCRDNYTDLVAIQNK | 281 |
| | | | | 104 | ◯ | — | IGGIWTWVGTNKS | 282 |
| 175 | basal cell adhesion molecule | gi\|31543106 | (isoform 1) | 377, 383 | ◯ | — | VAYLDPLELSEGKVLSLPLNSSAVVNCSVHGLPTPALR | 283 |
| | | gi\|61742797 | (isoform 2) | 377, 383 | ◯ | — | VAYLDPLELSEGKVLSLPLNSSAVVNCSVHGLPTPALR | 283 |
| 176 | AXL receptor tyrosine kinase | gi\|21536466 | (isoform 1) | 198 | ◯ | — | SLHVPGLNKTSSFSCEAHNAK | 284 |
| | | gi\|21536468 | (isoform 2) | 198 | ◯ | — | SLHVPGLNKTSSFSCEAHNAK | 284 |
| 177 | carbohydrate (chondroitin 4) | gi\|8922112 | | 209 | ◯ | — | EHVHNASAHLTFNK | 285 |
| | sulfotransferase 12 | | | 280 | ◯ | — | LYANHTSLPASAR | 286 |
| 178 | carboxypeptidase A4 | gi\|61743916 | | 260 | ◯ | — | SRNPGSSCIGADPNRNWNASFAGK | 287 |
| 179 | carcinoembryonic antigen-related cell | gi\|40255013 | | 224 | ◯ | — | RNDAGSYECEIQNPASANRSDPVTLNVLYGPDGPTISPSK | 288 |
| | adhesion molecule 6 (non-specific | | | | | | | |
| | cross reacting antigen) | | | | | | | |
| 180 | cell adhesion molecule 4 | gi\|21686977 | | 67 | ◯ | — | QTLFFNGTR | 289 |
| 181 | EGF-like-domain, multiple 3 | gi\|110347457 | | 961, 972 | ◯ | — | SACNCTAGAACDAVNGSCLCPAGR | 290 |
| 182 | fibroblast growth factor receptor 1 | gi\|105990522 | (isoform 1) | 264 | ◯ | — | SPHRPILQAGLPANKT | 291 |
| | | | | 296 | ◯ | — | HIEVNGSKIGPDNLPYVQILK | 292 |
| | | gi\|13186251 | (isoform 2) | 262 | ◯ | — | SPHRPILQAGLPANKT | 291 |
| | | | | 294 | ◯ | — | HIEVNGSKIGPDNLPYVQILK | 292 |
| | | gi\|13186234 | (isoform 3) | 175 | ◯ | — | SPHRPILQAGLPANKT | 291 |
| | | | | 207 | ◯ | — | HIEVNGSKIGPDNLPYVQILK | 292 |
| | | gi\|13186236 | (isoform 4) | 173 | ◯ | — | SPHRPILQAGLPANKT | 291 |
| | | | | 205 | ◯ | — | HIEVNGSKIGPDNLPYVQILK | 292 |
| | | gi\|13186238 | (isoform 5) | 175 | ◯ | — | SPHRPILQAGLPANKT | 291 |
| | | | | 207 | ◯ | — | HIEVNGSKIGPDNLPYVQILK | 292 |
| | | gi\|13186241 | (isoform 6) | 173 | ◯ | — | SPHRPILQAGLPANKT | 291 |
| | | | | 205 | ◯ | — | HIEVNGSKIGPDNLPYVQILK | 292 |
| | | gi\|13186249 | (isoform 9) | 264 | ◯ | — | SPHRPILQAGLPANKT | 291 |
| | | | | 296 | ◯ | — | HIEVNGSKIGPDNLPYVQILK | 292 |
| 183 | fibulin 1 | gi\|34734068 | (isoform A) | 535, 539 | ◯ | — | NCQDIDECVTGIHNCSINETCFNIQGGFR | 293 |
| | | gi\|34734064 | (isoform B) | 535, 539 | ◯ | — | NCQDIDECVTGIHNCSINETCFNIQGGFR | 293 |
| | | gi\|34734062 | (isoform C) | 535, 539 | ◯ | — | NCQDIDECVTGIHNCSINETCFNIQGGFR | 293 |
| | | gi\|34734066 | (isoform D) | 535, 539 | ◯ | — | NCQDIDECVTGIHNCSINETCFNIQGGFR | 293 |
| 184 | G protein-coupled receptor 126 | gi\|74048357 | (alpha 1) | 324 | ◯ | — | ILSNLSCNVK | 294 |
| | | | | 593, 600, 605 | ◯ | — | EANEVANQILNLTADGQNLTSANITNIVEQVKR | 295 |
| | | gi\|74048422 | (alpha 2) | 324 | ◯ | — | ILSNLSCNVK | 294 |
| | | | | 565, 572, 577 | ◯ | — | EANEVANQILNLTADGQNLTSANITNIVEQVKR | 295 |
| | | gi\|50355941 | (beta 1) | 324 | ◯ | — | ILSNLSCNVK | 294 |
| | | | | 593, 600, 605 | ◯ | — | EANEVANQILNLTADGQNLTSANITNIVEQVKR | 295 |
| | | gi\|74048403 | (beta 2) | 324 | ◯ | — | ILSNLSCNVK | 294 |
| | | | | 565, 572, 577 | ◯ | — | EANEVANQILNLTADGQNLTSANITNIVEQVKR | 295 |
| 185 | inducible T-cell co-stimulator ligand | gi\|27477039 | | 102 | ◯ | — | LFNVTPQDEQK | 296 |
| 186 | platelet-derived growth factor C | gi\|9994187 | | 55 | ◯ | — | IITVSTNGSIHSPR | 297 |
| 187 | plexin B1 | gi\|40254442 | | 1253 | ◯ | — | YTLDPNITSAGPTK | 298 |
| 188 | prosaposin | gi\|11386147 | (isoform a) | 80 | ◯ | — | SLPCDICKDVVTAAGDMLKDNATEEEILVYLEK | 299 |
| | | | | 101 | ◯ | — | TCDWLPKPNMSASCK | 300 |
| | | | | 426 | ◯ | — | NSTKQEILAALEK | 301 |
| | | gi\|110224476 | (isoform b) | 80 | ◯ | — | SLPCDICKDVVTAAGDMLKDNATEEEILVYLEK | 299 |
| | | | | 101 | ◯ | — | TCDWLPKPNMSASCK | 300 |
| | | | | 429 | ◯ | — | NSTKQEILAALEK | 301 |
| | | gi\|110224479 | (isoform c) | 80 | ◯ | — | SLPCDICKDVVTAAGDMLKDNATEEEILVYLEK | 299 |
| | | | | 101 | ◯ | — | TCDWLPKPNMSASCK | 300 |
| | | | | 428 | ◯ | — | NSTKQEILAALEK | 301 |
| 189 | semaphorin 7A | gi\|4504237 | | 105 | ◯ | — | VYLFDFPEGKNASVR | 302 |
| | | | | 157 | ◯ | — | HPSCWNLVNGTVVPLGEMR | 303 |
| 190 | UDP-Gal:betaGlcNAc | gi\|4502349 | | 385 | ◯ | — | RPPARPGPLSTANHTALRGSH | 304 |
| | beta 1,4-galactosyltransferase 3 | | | | | | | |
| 191 | UDP-GlcNAc:betaGal | gi\|5802984 | | 204 | ◯ | — | VAQPGINYALGTNVSYPNNLLR | 305 |
| | beta-1,3-N- | | | | | | | |
| | acetylglucosaminyltransferase 1 | | | 300 | ◯ | — | NELVQLYQVGEVRPFYYGLCTPCQAPTNYSR | 306 |
| 192 | UDP-GlcNAc:betaGal | gi\|9845238 | | 173 | ◯ | — | ESWGQESNAGNQTVVR | 307 |
| | beta-1,3-N- | | | | | | | |
| | acetylglucosaminyltransferase 2 | | | | | | | |
| 193 | intercellular adhesion molecule 1 | gi\|4557878 | | 267 | ◯ | — | LNPTVTYGNDSFSAK | 308 |
| 194 | haptoglobin-related protein | gi\|45580723 | | 126 | ◯ | — | MVSHHNLTTGATLINEQWLLTTAK | 309 |
| | | | | 149, 153 | ◯ | — | NLFLNHSENATAKDIAPTLTLYVGKK | 310 |
| 195 | lysosomal-associated membrane protein | gi\|112380628 | | 62 | ◯ | — | SGPKNMTFDLPSDATVVLNR | 311 |
| | 1 | | | 62, 76 | ◯ | — | SGPKNMTFDLPSDATVVLNRS | 312 |
| | | | | 121, 130 | ◯ | — | YSVQLMSFVYNLSDTHLFPNASSK | 313 |
| 196 | fibrinogen, gamma chain | gi\|70906437 | (A precursor) | 78 | ◯ | — | VDKDLQSLEDILHQVENKT | 314 |
| | | gi\|70906439 | (B precursor) | 78 | ◯ | — | VDKDLQSLEDILHQVENKT | 314 |
| 197 | leucine-rich alpha-2-glycoprotein 1 | gi\|16418467 | | 186 | ◯ | — | KLPPGLLANFTLLR | 315 |
| 198 | ADAM metallopeptidase with | gi\|21265034 | (isoform 1) | 667 | ◯ | — | YGEEYGNLTRPDITFTYFQPKPR | 316 |
| | thrombospondin type 1 motif, 13 | gi\|73695936 | (isoform 2) | 667 | ◯ | — | YGEEYGNLTRPDITFTYFQPKPR | 316 |
| | | gi\|21265043 | (isoform 3) | 636 | ◯ | — | YGEEYGNLTRPDITFTYFQPKPR | 316 |
| 199 | butyrylcholinesterase | gi\|4557351 | | 85 | ◯ | — | WSDIWNATK | 317 |
| 200 | complement component 1, | gi\|7705753 | | 146 | ◯ | — | RNPPMGGNVVIFDTVITNQEEPYQNHSGR | 318 |
| | q subcomponent, A chain | | | | | | | |
| 201 | polydom | gi\|148886654 | | 2876 | ◯ | — | VCLANGSWSGATPDCVPVR | 319 |
| | | | | 2992 | ◯ | — | CLSNGSWSGSSPSCLPCR | 320 |
| 202 | protease inhibitor 16 | gi\|70780384 | | 403, 409 | ◯ | — | SLPNFPNTSATANATGGR | 321 |
| 203 | alpha 1B-glycoprotein | gi\|21071030 | | 179 | ◯ | — | EGDHEFLEVPEAQEDVEATFPVHQPGNYSCSYR | 322 |
| 204 | apolipoprotein H | gi\|4557327 | | 162 | ◯ | — | VYKPSAGNNSLYR | 323 |
| | | | | 183, 193 | ◯ | — | DTAVFECLPQHAMFGNDTITCTTHGNWTKLPECR | 324 |
| | | | | 253 | ◯ | — | LGNWSAMPSCK | 325 |
| 205 | complement component 4A | gi\|67190748 | | 226 | ◯ | — | FSDGLESNSSTQFEVKK | 326 |
| | | | | 1328 | ◯ | — | GLNVTLSSTGR | 101 |
| 206 | mesothelin | gi\|53988378 | (isoform 1) | 488 | ◯ | ◯ | LAFQNMNGSEYFVK | 327 |
| | | gi\|53988380 | (isoform 2) | 496 | ◯ | — | LAFQNMNGSEYFVK | 327 |
| 207 | multimerin 1 | gi\|45269141 | | 114, 120 | ◯ | — | LQNLTLPTNASIK | 328 |
| | | | | 136 | ◯ | — | FNPGAESVVLSNSTLK | 329 |
| 208 | serine (or cysteine) proteinase | gi\|4502261 | | 128 | ◯ | — | LGACNDTLQQLMEVFKFDTISEK | 330 |
| | inhibitor, | | | 224 | — | ◯ | AAINKWVSNKTEGR | 331 |
| | clade C (antithrombin), member 1 | | | | | | | |
| 209 | fibrinogen, beta chain | gi\|70906435 | | 394 | ◯ | ◯ | GTAGNALMDGASQLMGENRT | 332 |
| 210 | major histocompatibility complex, | gi\|52630342 | | 110 | ◯ | — | GYYNQSEDGSHTLQR | 333 |
| | class I, C | | | | | | | |
| 211 | oxygen regulated protein | gi\|5453832 | | 830 | ◯ | — | LSALDNLLNHSSMFLK | 334 |
| | | | | 862, 869 | ◯ | — | VINETWAWKNATLAEQAK | 335 |
| | | | | 922, 931 | ◯ | — | DKNGTRAEPPLNASASDQGEK | 336 |
| 212 | cathepsin B | gi\|22538431 | | 38 | ◯ | — | NTTWQAGHNFYNVDMSYLKR | 337 |
| | | gi\|22538433 | | | | | | |
| | | gi\|22538435 | | | | | | |
| | | gi\|22538437 | | | | | | |
| | | gi\|4503139 | | | | | | |
| 213 | alpha-galactosidase A | gi\|4504009 | | 215 | ◯ | — | SIVYSCEWPLYMWPFQKPNYTEIR | 338 |
| 214 | ADAM metallopeptidase domain 9 | gi\|4501915 | (isoform 1) | 125 | ◯ | — | GYVEGVHNSSIALSDCFGLR | 339 |
| | | | | 144, 154 | ◯ | — | GLLHLENASYGIEPLQNSSHFEHIIYR | 340 |
| | | gi\|54292121 | (isoform 2) | 125 | ◯ | — | GYVEGVHNSSIALSDCFGLR | 339 |
| | | | | 144, 154 | ◯ | — | GLLHLENASYGIEPLQNSSHFEHIIYR | 340 |
| 215 | calcium activated nucleotidase 1 | gi\|20270339 | | 88 | ◯ | — | LGQAPANWYNDTYPLSPPQRTPAGIR | 341 |
| 216 | serine protease inhibitor, Kunitz | gi\|10863909 | | 94 | ◯ | — | KCATVTENATGDLATSR | 342 |
| | type, 2 | | | | | | | |
| 217 | tripeptidyl-peptidase I | gi\|5729770 | | 443 | ◯ | — | FLSSSPHLPPSSYFNASGR | 343 |
| 218 | angiopoietin-like 4 protein | gi\|21536398 | (isoform a) | 177 | — | ◯ | LPEMAQPVDPAHNVSR | 344 |
| 219 | olfactomedin-like 3 | gi\|9910270 | | 177 | — | ◯ | IYVLDGTQNDTAFVFPR | 345 |
| 220 | olfactomedin-like 2A | gi\|116014339 | | 184 | — | ◯ | HYENHSAIMLGIKK | 346 |
| 221 | aspartate beta-hydroxylase | gi\|14589866 | (isoform a) | 452 | — | ◯ | LVQLFPNDTSLK | 347 |
| 222 | galactosidase, beta 1-like | gi\|40255043 | | 486 | — | ◯ | LSFGSNSSDFK | 348 |
| 223 | neuronal cell adhesion molecule | gi\|81158224 | (isoform B) | 842 | — | ◯ | VNVVNSTLAEVHWDPVPLK | 349 |
| 224 | semaphorin 3C | gi\|5454048 | | 585 | — | ◯ | NNTTFLECAPK | 350 |
| 225 | adlican | gi\|139948432 | | 2415 | — | ◯ | SDSGNYTCLVR | 351 |
| 226 | amiloride binding protein 1 | gi\|73486661 | | 538 | — | ◯ | LENITNPWSPR | 352 |
| 227 | astacin-like metalloendopeptidase (M12 | gi\|66392154 | | 266 | — | ◯ | WNLSASDITR | 353 |
| | family) | | | | | | | |
| 228 | carbonic anhydrase XI | gi\|9951923 | | 108 | — | ◯ | HVSFLPAPRPVVNVSGGPLLYSHR | 354 |
| 229 | follistatin-like 3 glycoprotein | gi\|5031701 | | 73 | — | ◯ | AECCASGNIDTAWSNLTHPGNK | 355 |
| 230 | glycosylphosphatidylinositol specific | gi\|29171717 | (isoform 1) | 568, 591 | — | ◯ | LNVEAANWTVRGEEDFSWFGYSLHGVTVDNRT | 356 |
| | phospholipase D1 | | | | | | | |
| 231 | H factor (complement)-like 3 | gi\|5031695 | | 126 | — | ◯ | LQNNENNISCVER | 357 |
| 232 | inhibin beta B subunit | gi\|9257225 | | 93 | — | ◯ | GRPNITHAVPK | 358 |
| 233 | latent transforming growth factor | gi\|18497288 | | 349 | — | ◯ | LNSTHCQDINECAMPGVCR | 359 |
| | beta binding protein 3 | | | | | | | |
| 234 | legumain | gi\|56682962 | | 263, 272 | — | ◯ | SHTNTSHVMQYGNKTISTMK | 360 |
| 235 | serine (or cysteine) proteinase | gi\|21361302 | | 108 | — | ◯ | SQILEGLGFNLTELSESDVHR | 361 |
| | inhibitor, | | | | | | | |
| | clade A (alpha-1 antiproteinase, | | | | | | | |
| | antitrypsin), member 4 | | | | | | | |
| 236 | spondin 1, extracellular matrix | gi\|110347423 | | 214 | — | ◯ | LTFYGNWSEK | 362 |
| | protein | | | | | | | |
| 237 | ABI gene family, member 3 (NESH) | gi\|33667044 | | 44 | — | ◯ | VHINTTSDSILLK | 363 |
| | binding protein | | | | | | | |
| 238 | asporin (LRR class 1) | gi\|41350214 | | 282 | — | ◯ | LGLGNNKITDIENGSLANIPR | 364 |
| 239 | carboxypeptidase X, member 1 | gi\|9994201 | | 318 | — | ◯ | QVQEQCPNITR | 365 |
| 240 | chordin-like 1 | gi\|34147715 | | 291 | — | ◯ | AFGIVECVLCTCNVTK | 366 |
| 241 | immunoglobulin J chain | gi\|21489959 | | 71 | — | ◯ | IIVPLNNRENISDPTSPLR | 367 |
| 242 | desmoglein 2 | gi\|116534898 | | 462 | — | ◯ | YVQNGTYTVK | 368 |
| 243 | slit-like 2 | gi\|88702793 | | 117 | — | ◯ | LHEITNETFR | 369 |
| 244 | paraoxonase 1 | gi\|19923106 | | 324 | — | ◯ | VTQVYAENGTVLQGSTVASVYK | 370 |
| 245 | cathepsin C | gi\|4503141 | (isoform a) | 276 | — | ◯ | ILTNNSQTPILSPQEVVSCSQYAQGCEGGFPYLIAGK | 371 |
| 246 | ceroid-lipofuscinosis, neuronal 5 | gi\|5729772 | | 304 | — | ◯ | NIETNYTR | 372 |
| 247 | claudin domain containing 1 protein | gi\|11096340 | (isoform a) | 42 | — | ◯ | SPVQENSSDLNK | 373 |
| 248 | fibroblast growth factor receptor-like | gi\|51988910 | | 293 | — | ◯ | RVEYGAEGRHNSTIDVGGQK | 374 |
| | 1 | | | | | | | |
| 249 | oncostatin M receptor | gi\|4557040 | | 176 | — | ◯ | NIQNNVSCYLEGK | 375 |
| 250 | semaphorin 3A | gi\|5174673 | | 590 | — | ◯ | IIYGVENSSTFLECSPK | 376 |
| 251 | superoxide dismutase 3, extracellular | gi\|118582275 | | 107 | — | ◯ | AKLDAFFALEGFPTEPNSSSR | 377 |
| 252 | tissue inhibitor of metalloproteinase | gi\|4507513 | | 207 | — | ◯ | GWAPPDKSIINATDP | 378 |
| | 3 | | | | | | | |
| 253 | laminin alpha 2 subunit | gi\|28559088 | (isoform a) | 2657 | — | ◯ | YMQNLTVEQPIEVK | 379 |
| 254 | serine (or cysteine) proteinase | gi\|21361195 | | 262 | — | ◯ | VVGVPYQGNATALFILPSEGK | 380 |
| | inhibitor, | | | | | | | |
| | clade A (alpha-1 antiproteinase, | | | | | | | |
| | antitrypsin), member 5 | | | | | | | |
| 255 | periostin, osteoblast specific factor | gi\|5453834 | | 599 | — | ◯ | IFLKEVNDTLLVNELKSK | 381 |
| 256 | peroxidasin homolog | gi\|109150416 | | 1368 | — | ◯ | QGEHLSNSTSAFSTR | 382 |
| 257 | pregnancy specific beta-1-glycoprotein | gi\|4506175 | (isoform a) | 302 | — | ◯ | ILILPSVTRNETGPYQCEIR | 383 |
| | 6 | | | | | | | |
| 258 | glycoprotein hormones, alpha | gi\|4502787 | | 76 | — | ◯ | TMLVQKNVTSESTCCVAK | 384 |
| | polypeptide | | | | | | | |
| 259 | PTK7 protein tyrosine kinase 7 | gi\|15826840 | (isoform a) | 175 | — | ◯ | DGTPLSDGQSNHTVSSK | 385 |
| 260 | protein tyrosine phosphatase, | gi\|104487006 | (isoform 1) | 733 | — | ◯ | 726/KVEAEALNATAIR/738 | 386 |
| | receptor type, sigma | | | | | | | |
| 261 | unc5C | gi\|16933525 | | 236 | — | ◯ | LSDTANYTCVAK | 387 |
| 262 | latent transforming growth factor | gi\|110347437 | (isoform c) | 62 | — | ◯ | NATSVDSGAPGGAAPGGPGFR | 388 |
| | beta binding protein 4 | | | | | | | |

(2) The glycoprotein for an epithelial ovarian cancer diagnosis marker according to (1), wherein the glycan is a fucosylated glycan and/or a glycan comprising terminal N-acetylgal acto samine.

(3) The glycoprotein for an epithelial ovarian cancer diagnosis marker according to (1) or (2), wherein the glycan binds to AAL lectin and/or WFA lectin.

(4) The glycoprotein for an epithelial ovarian cancer diagnosis marker according to any of (1) to (3), wherein the epithelial ovarian cancer is at least one of clear cell carcinoma, mucinous carcinoma, serous carcinoma, and endometrioid carcinoma.

(5) The glycoprotein for an epithelial ovarian cancer diagnosis marker according to (4), wherein the protein is collagen type VI alpha 1, and the epithelial ovarian cancer is clear cell carcinoma or serous carcinoma.

(6) A fragment of a glycoprotein for an epithelial ovarian cancer diagnosis marker according to any of (1) to (5), comprising at least one glycan-linked asparagine residue at the glycosylation site shown in Table 1.

(7) A method for determining the presence or absence of epithelial ovarian cancer, comprising the steps of: detecting at least one glycoprotein for an epithelial ovarian cancer diagnosis marker according to any of (1) to (5) and/or at least one fragment of the glycoprotein for an epithelial ovarian cancer diagnosis marker according to (6) from a sample collected from a test subject; and determining that the test subject is cotracted with epithelial ovarian cancer when the glycoprotein for an epithelial ovarian cancer diagnosis marker and/or the fragment of the glycoprotein have been detected.

(8) The method according to (7), wherein the detection step comprises a glycoprotein enrichment step and a protein detection step.

(9) The method according to (7) or (8), wherein the glycoprotein for an epithelial ovarian cancer diagnosis marker and/or the fragment of the glycoprotein are detected using at least one glycan probe binding to the glycan.

(10) The method according to (9), wherein the glycan probe is a lectin, an antibody, or a phage antibody.

(11) The method according to (10), wherein the lectin is AAL or WFA.

(12) The method according to any of (7) to (11), wherein the sample is a body fluid, a cell, or a peritoneal lavage fluid.

(13) The method according to any of (7) to (12), wherein the epithelial ovarian cancer is at least one of clear cell carcinoma, mucinous carcinoma, serous carcinoma, and endometrioid carcinoma.

### Advantageous Effects of Invention

The epithelial ovarian cancer diagnosis marker and the method for determining the presence or absence of epithelial ovarian cancer according to the present invention enable the presence or absence of epithelial ovarian cancer to be determined conveniently, relatively inexpensively, and low invasively with high accuracy using a body fluid, a cell, or a peritoneal lavage fluid.

### BRIEF DESCRIPTION OF DRAWINGS

FIG. 1 shows results of selecting probe lectins. RMUG-S, RMG-I, RMG-II, RMG-V, and RTSG are cultured cells derived from epithelial ovarian cancer. Colo205 and Colo201 are cultured cells derived from colon cancer. KATO III is cultured cells derived from stomach cancer. FIG. 1A shows the results about WFA lectin selected as a probe lectin. FIG. 1B shows the results about AAL lectin also selected as a probe lectin.

FIG. 2 is a Western blot image showing the presence of collagen type VI alpha 1 (COL6α1) glycoprotein in peritoneal lavage fluids collected from clear cell adenocarcinoma patients, endometrioid adenocarcinoma patients, serous adenocarcinoma patients, and stomach cancer patients (two patients each). Each peritoneal lavage fluid was subjected to AAL lectin column chromatography to enrich and separate glycoproteins binding to AAL lectin, followed by the detection of COL6α1 with an anti-COL6α1 antibody.

FIG. 3 is a Western blot image showing the presence of LOXL2 (lysyl oxidase-like 2) glycoprotein in peritoneal lavage fluids collected from clear cell adenocarcinoma patients, endometrioid adenocarcinoma patients, serous adenocarcinoma patients, and stomach cancer patients (two patients each). Each peritoneal lavage fluid was subjected to WFA lectin column chromatography to enrich and separate glycoproteins binding to WFA lectin, followed by the detection of LOXL2 with an anti-LOXL2 antibody.

FIG. 4 is a Western blot image showing the presence of ceruloplasmin (CP) glycoprotein in peritoneal lavage fluids collected from clear cell adenocarcinoma patients, endometrioid adenocarcinoma patients, serous adenocarcinoma patients, and a stomach cancer patient (two patients each except for one stomach cancer patient). Each peritoneal lavage fluid was subjected to WFA lectin column chromatography to enrich and separate glycoproteins binding to WFA lectin, followed by the detection of CP with an anti-CP antibody.

FIG. 5 is a Western blot image showing the presence of SERPING1 glycoprotein in peritoneal lavage fluids collected from clear cell adenocarcinoma patients, endometrioid adenocarcinoma patients, serous adenocarcinoma patients, and stomach cancer patients (two patients each). Each peritoneal lavage fluid was subjected to WFA lectin column chromatography to enrich and separate glycoproteins binding to WFA lectin, followed by the detection of SERPING1 with an anti-SERPING1 antibody.

FIG. 6 is a Western blot image showing the presence of F12 (blood coagulation factor XII) glycoprotein in peritoneal lavage fluids collected from clear cell adenocarcinoma patients, endometrioid adenocarcinoma patients, serous adenocarcinoma patients, and stomach cancer patients (two patients each). Each peritoneal lavage fluid was subjected to WFA lectin column chromatography to enrich and separate glycoproteins binding to WFA lectin, followed by the detection of F12 with an anti-F12 antibody.

### DESCRIPTION OF EMBODIMENTS

1. Glycoprotein for Epithelial Ovarian Cancer Diagnosis Marker and Fragment thereof having Glycan

The first embodiment of the present invention provides a glycoprotein for an epithelial ovarian cancer diagnosis marker described in Table 1 and a fragment of the glycoprotein.

### 1-1. Glycoprotein for Epithelial Ovarian Cancer Diagnosis Marker

The “glycoprotein for an epithelial ovarian cancer diagnosis marker” of this embodiment is a glycoprotein represented by any of Protein #1 to #262 in Table 1, wherein, in its amino acid sequence, a glycan specific for epithelial ovarian cancer is linked to an asparagine residue at least at a position (counted from the initiating amino acid residue (initiating methionine) as the first position) represented by “Glycosylation site” in Table 1. For example, the glycoprotein of Protein #1 in Table 1 corresponds to collagen type VI alpha 1 protein (hereinafter, referred to as “COL6α1”) having a glycan-linked asparagine residue at least at position 212 on its amino acid sequence. In the case where a plurality of glycosylation sites per protein are described in Table 1, the glycan may be linked to at least one of these sites. In the case where two asparagine residues are described as to, for example, biglycan of Protein #2 having (positions 270 and 311) or complement component 4 (C4) binding protein alpha chain of Protein #15 (positions 506 and 528), each protein in which the glycan is linked to at least one of the asparagine residues suffices as the glycoprotein for an epithelial ovarian cancer diagnosis marker of the present invention. Hereinafter, in the present specification, such a glycosylated protein is referred to as a “glycoprotein”, and a base protein moiety excluding a glycan is referred to as a “core protein”.

In the table, “gi(ID)” represents the ID number of the core protein in each glycoprotein of this embodiment. A plurality of gi(ID) numbers registered for one core protein are all described in the table. Also, a plurality of possible isoforms of one core protein are indicated by isoform numbers together with their gi(ID) numbers in the table. In the case where the position of a glycosylation site counted from the initiating amino acid residue differs among the isoforms due to mRNA splicing or the like, the corresponding glycosylation site of each isoform is described in the table.

The glycan linked to the asparagine residue in the glycoprotein of this embodiment is not particularly limited as long as the glycan is specific for epithelial ovarian cancer. In this context, the “glycan specific for epithelial ovarian cancer” include, for example, a fucosylated glycan and/or a glycan comprising terminal N-acetylgalactosamine (hereinafter, referred to as “GalNAc”). These glycans can be identified using a lectin, an antibody, or a phage antibody that specifically recognizes and binds to each glycan. Examples of the lectin against the fucosylated glycan include Aleuria aurantia-derived AAL lectin and Lens culinaris-derived LCA lectin, which each specifically bind to thereto. Examples of the lectin against the terminal GalNAc include Wisteria floribunda-derived WFA lectin, which specifically binds thereto.

In Table 1, each glycoprotein for an epithelial ovarian cancer diagnosis marker confirmed to bind to AAL lectin or WFA lectin is indicated in circle (∘), while an unconfirmed one is indicated in dash (−).

The histological types of “epithelial ovarian cancer” are known to consist principally of clear cell carcinoma, mucinous carcinoma, endometrioid carcinoma, and serous carcinoma. At least one of these histological types can be diagnosed using the glycoprotein for an epithelial ovarian cancer diagnosis marker of this embodiment. For example, lysyl oxidase-like 2 (LOXL2) glycoprotein represented by Protein #28, ceruloplasmin (CP) glycoprotein represented by Protein #35, blood coagulation factor XII (F12) glycoprotein represented by Protein #68, and serpin peptidase inhibitor clade G(Cl inhibitor) member 1 (SERPING1) glycoprotein represented by Protein #42 in Table 1 each permit the diagnosis of all of the histological types, i.e., clear cell carcinoma, mucinous carcinoma, endometrioid carcinoma, and serous carcinoma (see Example 2 described below). In other words, these glycoproteins can serve as epithelial ovarian cancer diagnosis markers useful for determining whether or not a test subject has epithelial ovarian cancer, irrespective of the histological types. On the other hand, collagen type VI alpha 1 (COL6α1) glycoprotein represented by Protein #1 in Table 1 permits the diagnosis of clear cell carcinoma and serous carcinoma (see Example 2 described below). The clear cell carcinoma is a histological type that occurs with increased frequency in Japan compared with Western countries and results in poorer prognosis than that of the serous carcinoma, and is also known to be complicated by endometriosis with high frequency, as with the endometrioid carcinoma (Yoshikawa H. et al., 2000, Gynecol. Obstet., 1: 11-17). Thus, the COL6α1 glycoprotein can serve as a marker capable of determining whether or not the histological type of epithelial ovarian cancer complicated by endometriosis is clear cell carcinoma. Since the clear cell carcinoma has a high rate of reoccurrence and exhibits chemotherapy resistance, this histological type requires strict follow-up even for cases where their lesions have been detected early and thereby excised. Hence, a marker, such as the COL6α1 glycoprotein, which is capable of identifying the clear cell carcinoma can serve as a very useful epithelial ovarian cancer diagnosis marker.

### 1-2. Fragment of Glycoprotein for Epithelial Ovarian Cancer Diagnosis Marker

The “fragment of the glycoprotein for an epithelial ovarian cancer diagnosis marker” of this embodiment refers to an oligopeptide or polypeptide fragment consisting of a portion of the glycoprotein for an epithelial ovarian cancer diagnosis marker. This fragment comprises, in its amino acid sequence, at least one asparagine residue at the glycosylation site shown in Table 1, wherein the glycan specific for epithelial ovarian cancer described in the paragraph “1-1. Glycoprotein for epithelial ovarian cancer diagnosis marker” is linked to this asparagine residue.

The amino acid length of the fragment of the glycoprotein for an epithelial ovarian cancer diagnosis marker is not particularly limited and is preferably 5 to 100 amino acids, 8 to 80 amino acids, or 8 to 50 amino acids.

Hereinafter, the glycoprotein for an epithelial ovarian cancer diagnosis marker and the fragment of the glycoprotein for a marker are also collectively referred to as an “epithelial ovarian cancer diagnosis marker”.

Specific examples of the fragment of the glycoprotein for an epithelial ovarian cancer diagnosis marker include glycopeptides consisting of amino acid sequences represented by SEQ ID NOs: 1 to 388, wherein the glycan specific for epithelial ovarian cancer is linked to an asparagine residue corresponding to the glycosylation site shown in Table 1. The glycopeptides listed are fragments of glycoproteins for epithelial ovarian cancer diagnosis markers that were obtained by an IGOT method (described later) in identifying the glycoprotein for an epithelial ovarian cancer diagnosis marker of this embodiment. All of these fragments can be used to determine the presence or absence of epithelial ovarian cancer. In each amino acid sequence shown in Table 1, the underlined asparagine residue (N) represents a glycan-linked asparagine residue. In the case where a plurality of underlined asparagine residues exist in the amino acid sequence shown in Table 1, each glycopeptide in which the glycan is linked to at least one of the asparagine residues suffices as the fragment of the glycoprotein for an epithelial ovarian cancer diagnosis marker of the present embodiment.

### 2. Method for Determining Presence or Absence of Epithelial Ovarian Cancer

The second embodiment of the present invention provides a method for determining the presence or absence of epithelial ovarian cancer.

The method of this embodiment comprises a detection step and a confirmation step. Hereinafter, each step will be described specifically.

### 2-1. Detection Step

The “detection step” is the step of detecting at least one glycoprotein for an epithelial ovarian cancer diagnosis marker and/or at least one fragment of the glycoprotein, i.e., the epithelial ovarian cancer diagnosis marker(s), according to Embodiment 1 from a sample collected from a test subject. This step further comprises, if necessary, a glycoprotein enrichment step and a protein detection step.

In the present specification, the “test subject” refers to a person to be subjected to examination, i.e., a human donor of a sample described later. The test subject may be any patient having a certain disease or any normal individual. The test subject is preferably a person possibly having epithelial ovarian cancer or an epithelial ovarian cancer patient.

The “sample” refers to a part that is obtained from the test subject and subjected to the determination method of this embodiment. For example, a body fluid, a cell (including cell extracts), or a peritoneal lavage fluid applies to the sample.

The “body fluid” refers to a biological sample in a liquid state collected directly from the test subject. Examples thereof include blood (including serum, plasma, and interstitial fluid), lymph, ascitic fluid, pleural effusion, sputum, spinal fluid, lacrimal fluid, nasal discharge, saliva, urine, vaginal fluid, and seminal fluid. The sample is preferably a body fluid such as blood, lymph, or ascitic fluid, or a peritoneal lavage fluid obtained using saline. The body fluid or the peritoneal lavage fluid collected from the test subject may be used, if necessary, after pretreatment such as dilution or concentration or the addition of an anticoagulant such as heparin thereto, or may be used directly without such pretreatment. Alternatively, the cell may be disrupted by a method known in the art to obtain its extracts. For the method for preparing cell extracts, see methods described in, for example, McMamee M. G. 1989, Biotechniques, 7: 466-475 or Johnson B.H. et al., 1994, Biotechnology (N Y), 12: 1357-1360. The body fluid or the peritoneal lavage fluid may be collected on the basis of a method known in the art. For example, blood or lymph can be collected according to a blood collection method known in the art. Specifically, peripheral blood can be collected from the vein or the like of a peripheral site by injection. Alternatively, the ascitic fluid or the peritoneal lavage fluid can be collected by transabdominal ultrasound-guided aspiration steering around the intestinal tract, or collected by aspiration using a syringe or the like from the Douglas' pouch after intraperitoneal injection of approximately 100 mL of saline during laparotomy. As for the cell, cells to be subjected to examination can be surgically collected from an appropriate organ or tissue.

The body fluid or the peritoneal lavage fluid may be used immediately after the collection, or may be cryopreserved for a given period and then treated, if necessary, by thawing or the like before use. In this embodiment, in the case of using serum or the peritoneal lavage fluid, the epithelial ovarian cancer diagnosis marker can be detected typically using a volume of 10 μL to 100 μL.

The epithelial ovarian cancer diagnosis marker to be detected in this step may be any epithelial ovarian cancer diagnosis marker described in Table 1. One epithelial ovarian cancer diagnosis marker may be detected, or two or more epithelial ovarian cancer diagnosis markers may be detected. Each individual epithelial ovarian cancer diagnosis marker can be sufficiently detected by the detection of at least one glycan-linked asparagine residue at a glycosylation site shown in Table 1 in a glycoprotein for this epithelial ovarian cancer diagnosis marker.

The method for detecting the epithelial ovarian cancer diagnosis marker may be any method without particular limitations as long as the method is known in the art and is capable of detecting the glycoprotein. At least one glycan probe that binds to the glycan in each epithelial ovarian cancer diagnosis marker can be used in the detection.

In the present specification, the “glycan probe” refers to a determinant that specifically recognizes a particular glycan and/or glycoconjugate such as a glycoprotein and binds thereto. Examples thereof include lectins, antibodies, and phage antibodies. When the glycan probe is a lectin, examples of the lectin that may be used in this step include AAL lectin, LCA lectin, and WFA lectin.

Specifically, the detection method that can be used is a method comprising, in combination, for example, a glycoprotein enrichment step of using a glycan probe specifically binding to the glycan in each epithelial ovarian cancer diagnosis marker to selectively enrich glycoproteins having the glycan, and a protein detection step of detecting the epithelial ovarian cancer diagnosis marker using an antibody or the like specific for its core protein. More specifically, the detection method is as follows, for example.

Glycoprotein enrichment step: A glycoprotein group contained in a peritoneal lavage fluid or a body fluid (e.g., serum) obtained from a test subject is separated using a glycan probe, for example, a lectin (hereinafter, in the present specification, referred to as lectin A for the sake of convenience), specifically binding to the glycan in the glycoproteins.

Protein detection step: Subsequently, a moiety other than the glycan specifically binding to lectin A, for example, the core protein, in the epithelial ovarian cancer diagnosis marker to be detected is detected using, an antibody or the like that specifically recognizes the core protein, for example, an anti-core protein antibody (hereinafter, in the present specification, referred to as “antibody B” for the sake of convenience).

As a result, the epithelial ovarian cancer diagnosis marker of interest having the glycan specifically binding to lectin A can be detected. These enrichment and detection steps for the core protein in the glycoprotein may be carried out in any order. For example, a protein enrichment step of enriching core proteins using the antibody B may be followed by the detection of the glycoprotein of interest using a glycan probe (e.g., lectin A) (glycoprotein detection step).

Alternatively, a method using an antibody that is specific for the epithelial ovarian cancer diagnosis marker having the glycan specifically binding to lectin A and recognizes both of its glycan and protein moieties as epitopes can be used for detecting the epithelial ovarian cancer diagnosis marker of interest. This method is convenient because the epithelial ovarian cancer diagnosis marker of interest, i.e., the epithelial ovarian cancer diagnosis marker having the glycan specifically binding to lectin A, contained in a peritoneal lavage fluid or serum obtained from a test subject can be detected by one step.

This method can be achieved by use of a lectin A-immobilized column or array, and means of detecting the epithelial ovarian cancer diagnosis marker, more specifically, for example, lectin-antibody sandwich ELISA, antibody-overlay lectin array method, lectin-overlay-antibody array method, mass spectrometry (including high-performance liquid chromatography-mass spectrometry (LC-MS), high-performance liquid chromatography-tandem mass spectrometry (LC-MS/MS), gas chromatography-mass spectrometry (GC-MS), gas chromatography-tandem mass spectrometry (GC-MS/MS), capillary electrophoresis-mass spectrometry (CE-MS), and ICP-mass spectrometry (ICP-MS)), immunoassay, enzymatic activity assay, capillary electrophoresis, gold colloid method, radioimmunoassay, latex agglutination immunoassay, fluorescent immunoassay, Western blotting, immunohistochemical method, surface plasmon resonance spectroscopy (SPR method), or quartz crystal microbalance (QCM) method, using an antibody against the epithelial ovarian cancer diagnosis marker, preferably a monoclonal or polyclonal antibody specific for the epithelial ovarian cancer diagnosis marker having the glycan specifically binding to lectin A. All of these methods are known in the art and can be carried out according to ordinary methods in the art. As specific examples, the lectin-antibody sandwich ELISA, the antibody-overlay lectin array method, and the lectin-overlay-antibody array method will be described below.

The lectin-antibody sandwich ELISA is based on the same fundamental principles as those of sandwich ELISA using two types of antibodies except that one of the antibodies in the sandwich ELISA is merely replaced by a lectin. Thus, this approach is also applicable to automatization using an existing automatic immunodetection apparatus. The point to be noted is only the reaction between antibodies and lectins to be used for sandwiching antigens. Each antibody has at least two N-linked glycans. When the lectins used recognize glycans on the antibodies, background noise occurs during sandwich detection due to the binding reaction therebetween. A possible method for preventing the generation of this noise signal involves modifying the glycan moieties on the antibodies or using only Fab fragments, which contain no such glycan moieties. For these purposes, approaches known in the art can be used. The method for modifying the glycan moieties is described in, for example, Chen S. et al., 2007, Nat. Methods, 4: 437-44 or Comunale M.A. et al., 2009, J. Proteome Res., 8: 595-602. The method using Fab fragments is described in, for example, Matsumoto H. et al., 2010, Clin. Chem. Lab. Med., 48: 505-512.

The antibody-overlay lectin array method is a method using a lectin microarray. The lectin array refers to a substrate on which plural types of lectins differing in specificity are immobilized in parallel (i.e., in the form of array) as glycan probes. The lectin array can realize concurrent analysis on the types of lectins interacted with analyte glycoconjugates and the degrees of these interactions. The fundamental principles of this lectin microarray technique are described in, for example, Kuno A. et al. 2005, Nat. Methods 2: 851-856. A lectin array in which 45 types of lectins are immobilized on a substrate is commercially available as LecChip from GP BioSciences Ltd. and may therefore be used. In the antibody-overlay lectin array method, a fluorescent group or the like can be introduced indirectly into the sample of the test subject via an antibody, and concurrent multisample analysis can be achieved conveniently and quickly using the lectin array. The specific procedures of this method are described in Kuno A. et al., 2009, Mol. Cell Proteomics 8: 99-108, Jun Hirabayashi et al., 2007, Experimental Medicine, extra number “Study on Cancer Diagnosis at Molecular Level—Challenge to Clinical Application”, Yodosha Co., Ltd., Vol. 25 (17): 164-171, and Atsushi Kuno et al., 2008, Genetic Medicine MOOK No. 11, pp. 34-39, Medical Do, Inc.

For example, glycan moieties in glycoproteins in the sample of the test subject are specifically recognized by lectins on the lectin microarray. Thus, antibodies against the core protein moieties thereof can be overlaid on the glycoproteins to thereby specifically and highly sensitively detect the glycoproteins without labeling the test glycoproteins or highly purifying them.

The lectin overlay-antibody microarray method is a method using, instead of the lectin microarray for the antibody-overlay lectin array method, an antibody array in which antibodies against core proteins are immobilized in parallel (i.e., in the form of array) on a substrate such as a glass substrate. This method is based on the same fundamental principles as those of the antibody-overlay lectin array method except that the relationship between the lectins and the antibodies is merely reversed. The method, however, requires the same number of antibodies as the number of epithelial ovarian cancer diagnosis markers to be examined and also requires determining lectins in advance for detecting the alteration of glycans.

In these various detection methods, a commercially available polyclonal or monoclonal antibody that specifically recognizes the epithelial ovarian cancer diagnosis marker used or its core protein may be used. If such an antibody is not easily obtainable, this antibody can be prepared, for example, by a method given below.

First, the polyclonal antibody against the anti-epithelial ovarian cancer diagnosis marker glycopeptide can be prepared using a method well known in the art. Specifically, an adjuvant is added to the glycoprotein for an epithelial ovarian cancer diagnosis marker or the glycopeptide as an antigen to be detected. The glycopeptide for an epithelial ovarian cancer diagnosis marker containing glycosylation site(s) (asparagine residue(s)) may be synthesized and used as an antigen. Examples of the adjuvant include complete Freund's adjuvant and incomplete Freund's adjuvant. These adjuvants may be used as a mixture. The antigen may be inoculated, together with the adjuvant, to an antibody-producing animal to thereby boost antibody production. Alternatively, this peptide may be covalently bonded to commercially available keyhole limpet hemocyanin (KLH) or the like and inoculated to an antibody-producing animal. In this operation, granulocyte-macrophage colony stimulating factor (GM-CSF) may also be administered to the animal simultaneously therewith to thereby boost antibody production. Examples of the antibody-producing animal that can be used in antigen inoculation include mammals, for example, mice, rats, horses, monkeys, rabbits, goats, and sheep. The immunization can employ any of existing methods and is performed mainly by intravenous injection, hypodermic injection, intraperitoneal injection, or the like. The interval between immunization doses is not particularly limited and is an interval of several days to several weeks, preferably 4 to 21 days.

2 to 3 days after the final immunization date, whole blood is collected from the immunized animal. After serum separation, the polyclonal antibody can be prepared.

Alternatively, for example, the monoclonal antibody against the anti-epithelial ovarian cancer marker glycopeptide can be prepared by the method of Kohler & Milstein (Nature, 256: 495-497 (1975)). For example, antibody-producing cells obtained from the antigen-immunized animal are fused with myeloma cells to prepare hybridomas. From the obtained hybridomas, clones producing the anti-epithelial ovarian cancer diagnosis marker glycopeptide monoclonal antibody can be selected to thereby prepare the monoclonal antibody.

Specifically, 2 to 3 days after the final immunization date in the preparation of the polyclonal antibody, antibody-producing cells are collected. Examples of the antibody-producing cells include spleen cells, lymph node cells, and peripheral blood cells.

A cell line that is derived from any of various animals (e.g., mice, rats, and humans) and is generally obtainable by those skilled in the art is used as the myeloma cells to be fused with the antibody-producing cells. The cell line used is a drug-resistant cell line that cannot survive in a selective medium (e.g., HAT medium) in an unfused state, but can characteristically survive therein only in a fused state. In general, 8-azaguanine-resistant line is used. This cell line is deficient in hypoxanthine-guanine-phosphoribosyl transferase and cannot grow in a hypoxanthine-aminopterin-thymidine (HAT) medium.

The myeloma cells have already been known in the art, and various cell lines can be used preferably, for example, P3 (P3x63Ag8.653) (Kearney J. F. et al., 1979, J. Immunol., 123: 1548-1550), P3x63Ag8U.1 (Yelton D.E. et al., 1978, Curr. Top. Microbiol. Immunol., 81: 1-7), NS-1 (Kohler G. et al., 1976, Eur. J. Immunol., 6: 511-519), MPC-11 (Margulies D. H. et al., 1976, Cell, 8: 405-415), SP2/0 (Shulman M. et al., 1978, Nature, 276: 269-270), FO (de St. Groth S. F. et al., 1980, J. Immunol. Methods, 35: 1-21), 5194 (Trowbridge I.S. 1978, J. Exp. Med., 148: 313-323), and 8210 (Galfre G. et al., 1979, Nature, 277: 131-133).

Next, the myeloma cells and the antibody-producing cells are fused with each other. This cell fusion is performed by the contact between the myeloma cells and the antibody-producing cells at a mixing ratio of 1:1 to 1:10 at 30 to 37° C. for 1 to 15 minutes in the presence of a fusion promoter in a medium for animal cell culture such as MEM, DMEM, or RPMI-1640 medium or in a commercially available medium for cloning or cell fusion. A fusion promoter or a fusion virus, such as polyethylene glycol or polyvinyl alcohol having an average molecular weight of 1,000 to 6,000 or Sendai virus can be used for promoting the cell fusion. Alternatively, the antibody-producing cells and the myeloma cells may be fused with each other using a commercially available cell fusion apparatus based on electrical stimulation (e.g., electroporation).

After the cell fusion, hybridomas of interest are selected from the fused cells. Examples of the method therefor include a method using the selective growth of the cells in a selective medium. Specifically, the cell suspension is diluted with an appropriate medium and then seeded over a microtiter plate. A selective medium (e.g., HAT medium) is added to each well, and the cells are subsequently cultured with the selective medium appropriately replaced by a fresh one. As a result, the cells that have grown can be obtained as hybridomas.

The screening of the hybridoma is performed by, for example, a limiting dilution method or a fluorescence excitation method using a cell sorter. Finally, monoclonal antibody-producing hybridomas are obtained. Examples of the method for obtaining the monoclonal antibody from the obtained hybridomas include ordinary cell culture and ascitic fluid formation methods.

### 2-2. Confirmation Step

The “confirmation step” is the step of confirming the test subject with epithelial ovarian cancer when the epithelial ovarian cancer diagnosis marker has been detected in the detection step from the sample collected from the test subject.

The detection of the epithelial ovarian cancer diagnosis marker can be confirmed on the basis of whether or not the epithelial ovarian cancer diagnosis marker of interest has been detected as a result of conducting the detection method described in the detection step. When the determinant (e.g., glycan probe) has high specificity for the epithelial ovarian cancer diagnosis marker and exhibits no cross reactivity (i.e., when the determinant is an antibody), accurate diagnosis can be achieved by merely confirming the presence or absence of this detection.

On the other hand, when the determinant has relatively low specificity leading to the detection of other glycoproteins, etc. in addition to the epithelial ovarian cancer diagnosis marker to be detected (i.e., when detection background is relatively high), accurate diagnosis cannot be achieved by merely confirming the presence or absence of the detection. In this case, the presence or absence of epithelial ovarian cancer may be determined on the basis of a statistically significant difference in the amount of the target epithelial ovarian cancer diagnosis marker detected in the test subject compared with that in a normal individual. In this context, the “normal individual” is a person who has been shown to have no epithelial ovarian cancer, preferably a healthy person without any disease, more preferably a person similar in biological condition to the test subject, for example, a person having the same or similar sex, age, body weight, constitution (allergy, etc.), anamnesis, birth experience, or the like as that of the test subject.

The results of quantifying the epithelial ovarian cancer diagnosis marker by the detection method described in the detection step can be used as the amount of the epithelial ovarian cancer diagnosis marker detected. In this case, a protein known in the art expected to have no quantitative difference between the samples of the test subject and the normal individual can be used as an internal control to correct the quantification results of the test subject and the normal individual. Thus, the amount of the epithelial ovarian cancer diagnosis marker detected can be obtained more accurately. Examples of the protein for such an internal control include albumin.

The term “statistically significant” means that the statistical processing of the quantitative difference in the epithelial ovarian cancer diagnosis marker to be detected contained in respective samples collected from the test subject and the normal individual shows a significant difference between the samples. Specifically, examples of the significant difference include difference with a significance level smaller than 5%, 1%, or 0.1%. A testing method known in the art capable of determining the presence or absence of significance can be appropriately used as a testing method for the statistical processing without particular limitations. For example, the student's t test or multiple comparison test method can be used (Kanji Suzuki, Toukeigaku No Kiso (Basic of Statistics in English); and Yasushi Nagata, et al., Toukeiteki Tajyuhikakuhou No Kiso (Basic of Statistical Multiple Comparison Method in English)). The term “statistically significant difference” specifically means that the obtained value is higher or lower than a cutoff value defined as a value capable of separating patients from normal individuals such that sensitivity and specificity set by a routine method in multiple-specimen analysis are optimized.

Examples of specific methods for determining the presence or absence of epithelial ovarian cancer by the detection of the epithelial ovarian cancer diagnosis marker include methods given below.

When any one glycoprotein for an epithelial ovarian cancer diagnosis marker described in Table 1 or any one fragment of the glycoprotein, i.e., the epithelial ovarian cancer diagnosis marker, has been detected alone, the test subject as a donor of the sample is confirmed with epithelial ovarian cancer of type diagnosable with the epithelial ovarian cancer diagnosis marker. When no such marker has been detected, the test subject is confirmed not to have at least the epithelial ovarian cancer of type diagnosable with the epithelial ovarian cancer diagnosis marker. Specifically, when the LOXL2 glycoprotein represented by Protein #28 in Table 1, for example, has been detected in the sample, the test subject as a donor of the sample can be confirmed with epithelial ovarian cancer. Alternatively, when the COL6α1 glycoprotein represented by Protein #1 in Table 1 has been detected in the sample, the test subject as a donor of the sample can be confirmed with clear cell carcinoma or serous carcinoma or confirmed to highly possibly have the tumor.

Also, two or more epithelial ovarian cancer diagnosis markers described in Table 1 may be used in the detection. When these markers have been detected, the test subject as a donor of the sample is confirmed with epithelial ovarian cancer of type diagnosable with each of the epithelial ovarian cancer diagnosis markers. When no such markers have been detected, the test subject is confirmed not to have at least the epithelial ovarian cancer of type diagnosable with each of the epithelial ovarian cancer diagnosis markers. Specifically, for example, the LOXL2 glycoprotein represented by Protein #28 and the CP glycoprotein represented by Protein #35 in Table 1 are each used as epithelial ovarian cancer diagnosis markers in detection. When both of them have been detected, the test subject as a donor of the sample can be confirmed with epithelial ovarian cancer or confirmed to very highly possibly have the cancer. On the other hand, when the LOXL2 glycoprotein has been detected but the CP glycoprotein has not been detected, the results of detecting the LOXL2 glycoprotein are false-positive or the results of detecting the CP glycoprotein are false-negative. In this case, redetection, such as an attempt to detect a different epithelial ovarian cancer diagnosis marker can be confirmed to be necessary. Alternatively, the LOXL2 glycoprotein and the COL6α1 glycoprotein represented by Protein #1 are each used as epithelial ovarian cancer diagnosis markers in detection. When both of them have been detected, the test subject as a donor of the sample can be confirmed with epithelial ovarian cancer whose histological type is clear cell carcinoma when complicated by endometriosis. On the other hand, when the LOXL2 glycoprotein has been detected but the COL6α1 glycoprotein has not been detected, the test subject as a donor of the sample can be confirmed with epithelial ovarian cancer whose histological type is neither clear cell carcinoma nor serous carcinoma. Such detection of two or more epithelial ovarian cancer diagnosis markers is preferred as the method for determining the presence or absence of epithelial ovarian cancer according to this embodiment, because this method achieves more highly accurate diagnosis with lower false-positive and false-negative rates than those of the detection of any one epithelial ovarian cancer diagnosis marker alone and also can determine the presence or absence of epithelial ovarian cancer as well as its histological type.

### EXAMPLES

### Example 1

### Selection of Epithelial Ovarian Cancer Diagnosis Marker

1. Selection of Probe Lectin on Lectin Microarray using Culture Supernatant (Method)

### (1) Preparation of Culture Supernatants of Epithelial Ovarian Cancer, Stomach Cancer, and Colon Cancer Cell Lines

Five epithelial ovarian cancer cell lines (RMG-I, RMG-II, RTSG, RMG-V, and RMUG-S) were separately cultured using a medium containing 90% Ham F12 and 10% FBS (PS⁺: penicillin⁺ & streptomycin), while one stomach cancer cell line (KATO III) and two colon cancer cell lines (Colo201 and Colo205) were separately cultured as non-epithelial ovarian cancer cell lines using a medium containing 90% RPMI-1640 and 10% FBS (PS). In this operation, RMG-I, RMG-V, RMUG-S, RMG-II, and RTSG were each cultured in a dish having a diameter of 14 cm until reaching 80 to 90% confluency. After removal of the FBS-containing medium by suction, the cells of each cell line were washed seven times with 10 mL/dish of a non-supplemented medium (FBS⁻, PS), then supplemented with 30 mL of the same medium, and cultured for 48 hours. Alternatively, KATO III, Colo201, and Colo205 were each adjusted to 1 x 10⁷ cells per dish having a diameter of 14 cm. The cells of each cell line were washed seven times by suspension through the addition of 10 mL of the non-supplemented medium and removal of the supernatant through centrifugation (1000 rpm, 5 minutes, room temperature), then seeded over a 14-cm dish, and cultured for 48 hours after addition of 30 mL of the non-supplemented medium. The supernatant of each cell line thus cultured was recovered by centrifugation (1000 rpm, 5 minutes, room temperature). The recovered supernatant was centrifuged again (3000 rpm, 5 minutes, room temperature), and the supernatant was recovered and cryopreserved at -80° C. The preserved sample was thawed before use, filtered through a 0.46-pm filter, and then used in subsequent analysis.

(2) Selection of Probe lectin on Lectin Microarray

Each culture supernatant thus prepared was concentrated and desalted using 2-D Clean-Up kit (GE Healthcare Japan Corp.). The obtained precipitates were lysed again in 20 μL of PBS. The protein concentration of each culture supernatant was measured using Micro BCA protein assay kit (Pierce Biotechnology, Inc.). Then, the culture supernatant was diluted with 10-fold with PBS. Proteins were collected in an amount corresponding to 100 ng in total, adjusted to 10 μL with PBSTx (PBS containing 1% Triton X-100), and then reacted at room temperature for 1 hour after addition of 20 ng of a fluorescent labeling reagent (Cy3-SE, GE Healthcare Japan Corp.). After the reaction, 90 μL of a glycine-containing buffer solution was added thereto, and the proteins were further reacted at room temperature for 2 hours to inactivate a redundant labeling reagent. The resulting fluorescently labeled glycoprotein solution was applied to a lectin microarray. The lectin microarray was used in which 3 spots each of 43 different lectins were immobilized. To attain the optimum comparative analysis for acquired binding signals, four dilutions series per sample were prepared and analyzed. Lectin binding reaction was performed at 20° C. for 12 hours. After the reaction, the sample solution on the array was removed, and the array was washed three times with a dedicated buffer solution. Then, signal intensity was measured using a scanner for the lectin microarray (GlycoStation™ Reader 1200, manufactured by GP BioSciences Ltd.). A true value was calculated by the subtraction of a background value, and a mean among 3 spots of each lectin was then calculated. When the largest signal intensity among all of the lectins was defined as a reference, relative values were determined and statistically processed as follows: after conversion of the calculated relative values to common logarithms, two separate groups, i.e., epithelial ovarian cancer and non-epithelial ovarian cancer (stomach cancer and colon cancer) groups, were confirmed by signal pattern analysis using the cluster analysis method and the principal component analysis method. Next, WFA lectin that allowed significant difference to be confirmed between these two groups was extracted by the t study. At the same time, AAL lectin that yielded a high detectable signal in all of the samples subjected to the lectin microarray analysis was extracted. These WFA and AAL lectins were selected as probes for use in subsequent analysis.

### (Results)

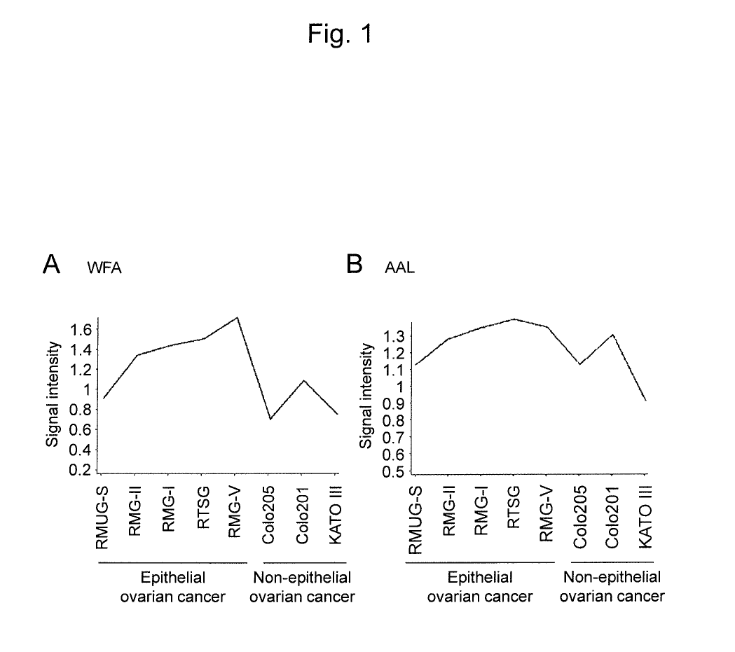

FIG. 1 shows the binding signal intensities of two types of lectins extracted by the preceding step to glycoproteins derived from each cancer cell line. FIGS. 1 A and 1B show the results about the WFA lectin and the AAL lectin, respectively. As shown in this drawing, the WFA lectin exhibited a high signal for the epithelial ovarian cancer cell lines, but a low signal for the colon cancer cell lines or the stomach cancer cell line as non-epithelial ovarian cancer cell lines. The AAL lectin exhibited a high signal for the epithelial ovarian cancer cell lines and the non-epithelial ovarian cancer cell lines, but a low signal for the stomach cancer cell line (KATO III). The WFA lectin and the AAL lectin had a low signal for RMUG-S, one of the epithelial ovarian cancer cell lines. This is due to the results of this experiment of analyzing the whole glycoproteins in order to select probe lectins and does not indicate behaviors related to the lectin-binding activities of the individual glycoproteins as shown in subsequent Examples. These results demonstrated that the WFA lectin and the AAL lectin can serve as probe lectins binding to the glycans of glycoproteins secreted from epithelial ovarian cancer cell lines.

### 2. Selection of Glycopeptide Marker by Glycoproteomics (IGOT-LC/MS Method) (Method)

### (1) Preparation of Peptide Sample

Sample proteins were prepared from culture supernatants of epithelial ovarian cancer cell lines, and peritoneal lavage fluids. The culture supernatants of epithelial ovarian cancer cell lines were prepared according to the method described in the paragraph 1.(1). The peritoneal lavage fluids used were peritoneal lavage fluids collected from 7 epithelial ovarian cancer patients (56 to 77 years old) (3 clear cell cancer patients, stages IIIC to IV; 1 endometrioid adenocarcinoma patient, stage IV; and 3 serous adenocarcinoma patients, IIIC to IV) during surgery at the Aichi Cancer Center Hospital. Trichloroacetic acid (TCA, 100% saturated aqueous solution) was added at a final concentration of 10% to each of the culture supernatants (1260 to 3300 mL) containing proteins in an amount corresponding to 3.6 to 7.6 mg in total and the peritoneal lavage fluids (0.3 to 300 mL) containing proteins in an amount corresponding to 8.2 to 15.4 mg in total. Each mixture was cooled on ice for 10 to 60 minutes to precipitate proteins. The precipitates were recovered by centrifugation at a high speed at 4° C., then suspended in ice-cold acetone, and washed twice for removal of TCA. A lysis buffer solution (containing 0.5 M tris-HC1 buffer solution (pH 8 to 8.5), 7 M guanidine hydrochloride, and 10 mM EDTA) was added to the obtained precipitates to adjust the protein concentration to 5 to 10 mg/mL, while the proteins were lysed therein to prepare sample proteins. In another method, each culture supernatant or peritoneal lavage fluid was concentrated using an ultrafiltration membrane having a molecular weight cutoff of 10,000 at 4° C. To this concentrate, a lysis buffer solution was added. The protein solution was filtered again to prepare sample proteins.

Subsequently, the sample proteins were subjected to centrifugation at a high speed at 4° C. to remove the residues. Each obtained supernatant was recovered as extracts. Dissolved oxygen was removed by nitrogen gas purging or spraying to the extracts. Then, dithiothreitol (DTT) in the form of a powder or dissolved in a small amount of a lysis buffer solution was added thereto in an amount equal to the protein weight. The mixture was reacted at room temperature for 1 to 2 hours under nitrogen gas purging or in a nitrogen gas atmosphere to reduce the disulfide bond. Subsequently, iodoacetamide for S-alkylation was added thereto in an amount of 2.5 times the protein weight. The mixture was reacted at room temperature for 1 to 2 hours in the dark. The reaction solution was dialyzed at 4° C. against a 50- to 100-fold amount of a buffer solution (10 mM ammonium bicarbonate buffer solution, pH 8.6) as an external solution. The external solution was replaced by a fresh one three to five times to remove the denaturant (guanidine hydrochloride) or an excess of the reagents. Although the partial precipitation of proteins was observed by these procedures, this suspension was subjected directly to protein quantification without recovery of the precipitates. Trypsin (sequencing grade or higher) having a weight of 1/100 to 1/50 of the protein amount was added thereto to digest the proteins overnight (approximately 16 hours) at 37° C. The sufficient progression of the digestion was confirmed by SD S-gel electrophoresis. Then, the reaction was terminated by the addition of phenylmethanesulfonyl fluoride (PMSF) at a final concentration of 5 mM. The obtained protein fragment (peptide) solutions were used as sample peptides.

### (2) Collection and Purification of Candidate Glycopeptide

The sample peptides were applied to probe lectin (AAL lectin and/or WFA lectin)-immobilized columns. After washing, glycopeptides were eluted by a method appropriate for the specificity of each lectin, i.e., using a buffer solution containing 5 mM fucose as to the AAL lectin and using a buffer solution containing 10 mM GaINAc as to the WFA lectin. To each obtained glycopeptide solution, an equal volume of ethanol and a 4-fold volume of 1-butanol were added, and the mixture was applied to a Sepharose column equilibrated in advance with water:ethanol:1-butanol (1:1:4 (v/v)). The column was washed with this equilibrating solvent, and glycopeptides were then eluted with 50% ethanol (v/v). Each glycopeptide fraction was transferred in small portions to a microtube containing 2 μL of glycerol and concentrated by repeated removal of water through centrifugation under reduced pressure. The obtained glycerol solutions of purified glycopeptides were used as glycopeptide samples.

### (3) Glycan Cleavage and Isotope-Coded Glycosylation Site-Specific Tagging (IGOT) Method

A necessary amount of a buffer solution was added to each of the glycopeptide samples, and the mixture was concentrated again by centrifugation under reduced pressure. Then, stable oxygen isotope-18 (¹⁸O)-labeled water (H₂¹⁸O) was added thereto to dissolve the concentrate (glycerol concentration: 10% or lower). Peptide-N-glycanase (glycopeptidase F, PNGase) prepared with labeled water was added thereto and reacted overnight at 37° C. This reaction causes the conversion of asparagine at the glycosylation site to aspartic acid, during which the oxygen isotope (¹⁸O) in the water is incorporated into the glycopeptide to label the glycopeptide.

### (4) LC/MS Shotgun Analysis of Labeled Peptide

The reaction solution containing the glycopeptide labeled by the IGOT method was diluted with 0.1% formic acid and subjected to LC/MS shotgun analysis. A nano-LC system based on a direct nano-flow pump was used for high-separation, high-reproducibility, and high-sensitivity detection. The injected glycopeptide sample was temporarily collected onto a trap column (reverse-phase C18 silica gel carrier) intended for desalting. After washing, glycopeptides were separated by the concentration gradient of acetonitrile using frit-less spray tip nano-columns (inside diameter: 150 μm×50 mL) packed with the same resins. The eluate was ionized via an electrospray interface and introduced directly into a mass spectrometer. The masses of the glycopeptides were analyzed by collision-induced dissociation (CID)-tandem mass spectrometry in a data-dependent mode in which two ions at the maximum were selected.

### (5) Search for Candidate Glycopeptide by MS/MS Ion Search Method

Thousands of MS/MS spectra thus obtained were individually smoothed and converted to centroid spectra to prepare peak lists. On the basis of this data, each detected glycopeptide was identified by the MS/MS ion search method using a protein amino acid sequence database. The search engine used was Mascot (Matrix Science Ltd.). The following parameters were used for search conditions: a fragmentation method used: trypsin digestion, the maximum number of missed cleavage: 2, fixed modification: carbamidomethylation of cysteine, variable modifications: deamination of an N-terminal amino group (N-terminal glutamine), oxidation of methionine, ¹⁸0-incorporating deamidation of asparagine (glycosylation site), error tolerance of MS spectrum: 500 ppm, and error tolerance of MS/MS spectrum: 0.5 Da.

### (6) Identification of Glycoprotein for Epithelial Ovarian Cancer Diagnosis Marker

The identification results of the glycopeptides obtained by search under the conditions described above were validated according to criteria (i) to (iv) given below to select glycopeptides that satisfied all of the conditions.

(i) The probability score (coincidence probability: Expectation value) of identification is 0.05 or less.

(ii) The number of fragment ions contributing to identification is 4 or more.

(iii) Error (ppm) is not significantly deviated from systematic error (mass error being 0.5 Da or less).

(iv) Each identified peptide has consensus sequence(s) with the number of Asn modifications (conversion to Asp and incorporation of ¹⁸O) equal to or fewer than the number of the consensus sequence(s).

### (Results)

The selected glycopeptides are shown in SEQ ID NOs: 1 to 388 as “Peptide sequence” in Table 1. On the basis of the amino acid sequences of these peptides, the whole amino acid sequences of core proteins in the corresponding glycoproteins for epithelial ovarian cancer diagnosis markers were identified from the amino acid sequence database NCBI-Refseq. As a result, 262 glycoproteins for epithelial ovarian cancer diagnosis markers were identified. The names of the core proteins in these glycoproteins for epithelial ovarian cancer diagnosis markers are shown in Table 1.

### Example 2

### Detection of Epithelial Ovarian Cancer using Epithelial Ovarian Cancer Diagnosis Marker

The detection of epithelial ovarian cancer using the epithelial ovarian cancer diagnosis markers selected and identified in Example 1 was tested.

### (Method)

(1) Fractionation of Peritoneal Lavage Fluid using Probe Lectin

The peritoneal lavage fluids used were obtained from ovarian cancer patients having clear cell carcinoma, mucinous carcinoma, serous carcinoma, or endometrioid carcinoma (two patients each), and from two stomach cancer patients with intraperitoneal progression proved by qPCR after recovery of peritoneal lavage fluids using a syringe or the like from the Douglas' pouch after intraperitoneal injection of approximately 100 mL of saline during surgery. The concentration of proteins contained in each peritoneal lavage fluid was measured by the BCA method, and the total amount of proteins was adjusted to equal levels among the samples, which were then subjected to the following AAL lectin or WFA lectin fractionation.

For the AAL fractionation, 2 mL Disposable polystyrene column (Pierce Biotechnology, Inc.) was packed with 0.5 mL of AAL-agarose (Vector Laboratories, Inc.) and washed with TBS (pH 8) in an amount of 10 times the amount of the resin. Then, each peritoneal lavage fluid (total protein amount: 0.25 mg) adjusted to 500 μL with TBS was applied to the column. While the sample was held in the column, the column was left standing at room temperature for overnight reaction. Then, the pass-through sample was recovered, and the column was washed with 10 mL of TBS, followed by elution with 1 mL of 50 mM fucose-TBS (fraction A-1). The column was further left standing at room temperature for 30 minutes for reaction, followed by the recovery of a fraction using 2.4 mL of an eluent (fraction A-2). Then, the column was washed with 4 mL of TBS. Then, the whole amount of the pass-through fraction thus recovered was applied again to the column. While the sample was held in the column, the column was left standing at room temperature for 4-hour reaction. After the reaction, the column was washed with 10 mL of TBS, followed by the recovery of a fraction using 0.6 mL of an eluent (fraction A-3). The column was further left standing at room temperature for 30 minutes for reaction, followed by the recovery of a fraction using 1.4 mL of an eluent (fraction A-4). The fractions A-1 to A-4 were pooled as AAL(+) fractions of the peritoneal lavage fluid.

For the WFA fractionation, 2 mL Disposable polystyrene column (Pierce Biotechnology, Inc.) was packed with 0.3 mL of WFA agarose (Vector Laboratories, Inc.) and washed in the same way as in AAL described above. Then, each peritoneal lavage fluid (total protein amount: 2.5 mg) adjusted to 500 μL with TBS was applied to the column and reacted at room temperature for 30 minutes. After the reaction, the column was washed with 6 mL of TBS, followed by elution with 0.18 mL of 200 mM lactose-TBS (fraction W-1). Next, the column after the elution of the fraction W-1 was left standing at room temperature for 30 minutes, followed by the recovery of a fraction using 0.72 mL of an eluent (fraction W-2). The recovered fractions W-1 and W-2 were pooled as WFA(+) fractions of the peritoneal lavage fluid.

### (2) Immunoblotting

Each sample thus pooled was concentrated using Amicon Ultra-154 centrifugal filter units (cutoff: 10 kDa, Millipore Corp.) and then immunoblotted using antibodies specifically binding to the core proteins in the glycoproteins for epithelial ovarian cancer diagnosis markers obtained in Example 1.

The following five glycoproteins for epithelial ovarian cancer diagnosis markers were randomly selected from among the 262 markers obtained in Example 1 as shown in Table 1 on the condition that antibodies specifically recognizing their core proteins were commercially available: collagen type VI alpha 1 protein (COL6α1) of Protein #1, lysyl oxidase-like 2 protein (LOXL2) of Protein #28, ceruloplasmin protein (CP) of Protein #35, SERPING1 protein of Protein #42, and blood coagulation factor XII protein (F12) of Protein #68 in Table 1. The antibodies used were an anti-COL6α1 polyclonal antibody (17023-1-AP; ProteinTech), an anti-LOXL2 polyclonal antibody (GTX105085; GeneTex Inc.), an anti-CP polyclonal antibody (A80-124A; Bethyl Laboratories, Inc.), an anti-SERPING1 monoclonal antibody (3F4-1D9, H00000710-M01; Abnova Corp.), and an anti-F12 antibody (B7C9, GTX21007; GeneTex Inc.). These antibodies were biotinylated using Biotin Labeling Kit-NH₂ (Dojindo Laboratories). The biotinylation was performed according to the protocol attached to the kit.

Each AAL(+) fraction or WFA(+) fraction of the peritoneal lavage fluid prepared in the paragraph (1) was developed by SDS-PAGE on a 10% acrylamide gel of XV PANTERA SYSYTEM (Maruko Shokai Co., Ltd.) and then transferred to a PVDF membrane (Bio-Rad Laboratories, Inc.) at 200 mA for 90 minutes. The blocking agent used was PBST (PBS supplemented with 0.1% Tween 20) containing 5% skimmed milk or 5% BSA dissolved therein. The membrane was blocked overnight at 4° C. and then washed three times with PBST for 10 minutes per run. Subsequently, the biotinylated antibodies were added as primary antibodies to the membrane and reacted at room temperature for 1 hour. In this operation, the anti-COL6α1 antibody was added to the AAL(+) fraction-transferred membrane, while the anti-LOXL2 antibody, the anti-CP antibody, the anti-SERPING1 antibody, and the anti-F12 antibody were added to the WFA(+) fraction-transferred membrane. After the reaction, three 10-minute washing runs with PBST were performed again. Then, the membrane was reacted at room temperature for 1 hour with HRP-conjugated streptavidin (1:3000 dilution, GE Healthcare Japan Corp.) as secondary antibodies against the biotinylated antibodies. After three 10-minute washing runs with PBST, the enzymatic reaction of HRP was caused using Western Lightning Chemiluminescence Reagent Plus (PerkinElmer, Inc.). Signals were detected using Amersham Hyperfilm ECL (GE Healthcare Japan Corp.) and subjected to comparative analysis.

### (Results)

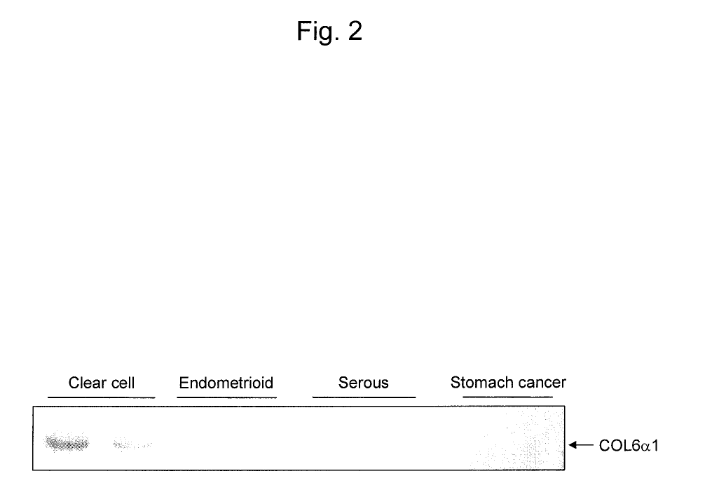

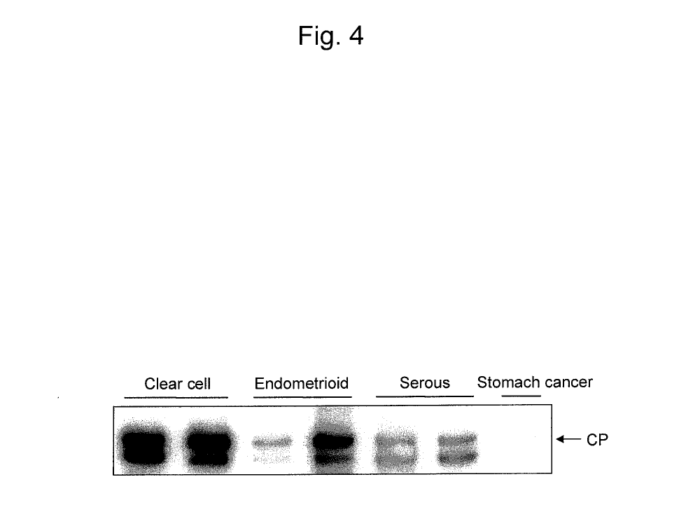

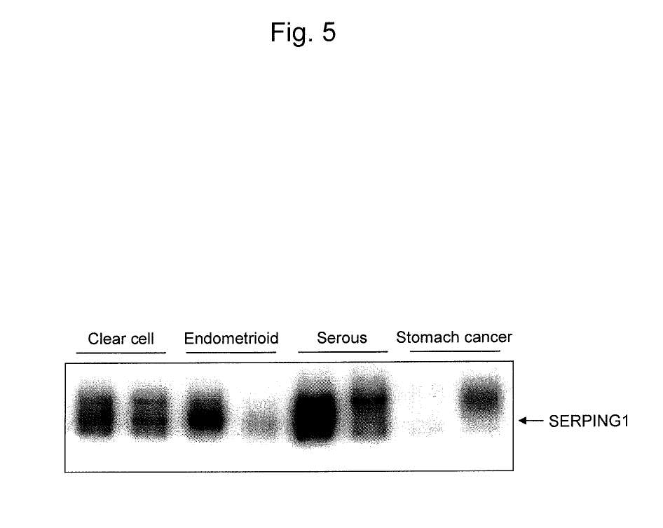

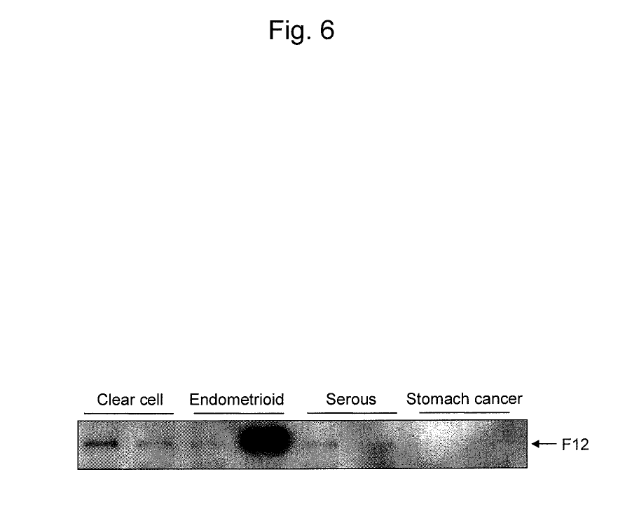

FIGS. 2 to 6 show the results of detecting each glycoprotein for an epithelial ovarian cancer diagnosis marker in the peritoneal lavage fluids obtained from the cancer patients.

As seen from the results of FIG. 2, COL6α1 was not detected in the patients with stomach cancer which was non-epithelial ovarian cancer. As for the epithelial ovarian cancer patients, this protein was detected in the clear cell adenocarcinoma patients and the serous adenocarcinoma patients, but hardly detected in the endometrioid adenocarcinoma patients. This demonstrated that COL6α1 can serve as a glycoprotein for an epithelial ovarian cancer diagnosis marker capable of selectively identifying clear cell carcinoma or serous carcinoma among epithelial ovarian cancer types. This protein was not detected in endometrioid carcinoma, which is often complicated by endometriosis, as with the clear cell carcinoma, demonstrating that the protein can serve as a marker glycoprotein capable of identifying clear cell carcinoma among types complicated by endometriosis.

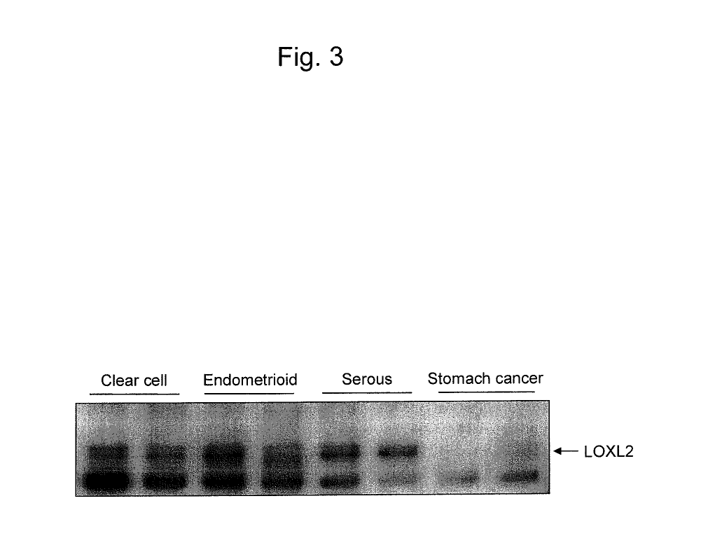

As seen from the results of FIGS. 3 to 6, LOXL2, CP, SERPING1, and F12 were all detected in the epithelial ovarian cancer patients having any of the histological types tested, but were not detected in the stomach cancer patients. This demonstrated that the LOXL2, CP, SERPING1, and F12 glycoproteins can each serve as a glycoprotein for an epithelial ovarian cancer diagnosis marker that permits the diagnosis of epithelial ovarian cancer, regardless of its histological type.

Since epithelial ovarian cancer was successfully detected in histological type-specific or -nonspecific manner using any of the 5 markers randomly selected from the glycoproteins for epithelial ovarian cancer diagnosis markers obtained in Example 1 as shown in Table 1, any glycoprotein described in Table 1 was shown to be able to serve as a glycoprotein for an epithelial ovarian cancer diagnosis marker.

All publications, patents, and patent applications cited herein are incorporated herein by reference in their entirety.

1-13. (canceled)

14. A method for determining the presence or absence of epithelial ovarian cancer in a test subject, comprising the steps of:
detecting at least one glycoprotein for an epithelial ovarian cancer diagnosis marker or a fragment thereof in a sample collected from a test subject, and
determining that the test subject is affected with epithelial ovarian cancer when the glycoprotein and/or the fragment of the glycoprotein have been detected;
wherein the glycoprotein and fragment thereof comprise at least one glycan-linked asparagine residue at a glycosylation site,
wherein the glycoprotein is selected from the group consisting of ceruloplasmin, lysyl oxidase-like 2, serpin peptidase inhibitor clade G member 1, coagulation factor XII, and collagen type VIα1,
wherein the glycan-linked asparagine residue in the amino acid of ceruloplasmin is at positions 138, 358, 397, and 762,
wherein the glycan-linked asparagine residue in the amino acid of lysyl oxidase-like 2 is at positions 288, 293, and 644,
wherein the glycan-linked asparagine residue in the amino acid of serpin peptidase inhibitor clade G member 1 is at positions 238, 253, and 352,
wherein the glycan-linked asparagine residue in the amino acid of coagulation factor XII is at position 249, and
wherein the glycan-linked asparagine residue in the amino acid of collagen type VIα1 is at position 212.

15. The method of claim 14, wherein the detecting step comprises a glycoprotein enrichment step and a protein detection step.

16. The method of claim 14, wherein the glycoprotein for an epithelial ovarian cancer diagnosis marker and/or the fragment thereof are detected by binding at least one glycan probe to the glycan.

17. The method of claim 16, wherein the glycan probe is a lectin, an antibody, or a phage antibody.

18. The method of claim 17, wherein the lectin is AAL or WFA.

19. The method of claim 14, wherein the sample is a body fluid, a cell, or a peritoneal lavage fluid.

20. The method of claim 14, wherein the epithelial ovarian cancer is at least one of clear cell carcinoma, mucinous carcinoma, serous carcinoma, and endometrioid carcinoma.


SEQUENCE LISTING (388 sequences)

SEQ ID NO: 1 - protein, 12 aa, Homo sapiens
RNFTAADWGQSR

SEQ ID NO: 2 - protein, 14 aa, Homo sapiens
MIENGSLSFLPTLR

SEQ ID NO: 3 - protein, 14 aa, Homo sapiens
LLQVVYLHSNNITK

SEQ ID NO: 4 - protein, 14 aa, Homo sapiens
KIVTATVNNSVLQK

SEQ ID NO: 5 - protein, 21 aa, Homo sapiens
NPVGLIGAENATGETDPSHSK

SEQ ID NO: 6 - protein, 28 aa, Homo sapiens
RIIQWLEAEIIPDGWFSKGSNYSEILDK

SEQ ID NO: 7 - protein, 17 aa, Homo sapiens
WYSLGKVPGNQTSTTLK

SEQ ID NO: 8 - protein, 36 aa, Homo sapiens
VQWRPQGTRGPWQEQIVSDPFLVVSNTSTFVPYEIK

SEQ ID NO: 9 - protein, 12 aa, Homo sapiens
THNLTDLSPHLR

SEQ ID NO: 10 - protein, 20 aa, Homo sapiens
SENYTYIWWLNGQSLPVSPR

SEQ ID NO: 11 - protein, 20 aa, Homo sapiens
ILILPSVTRNETGPYECEIR

SEQ ID NO: 12 - protein, 22 aa, Homo sapiens
ILILPSVTRNETGPYQCEIQDR

SEQ ID NO: 13 - protein, 14 aa, Homo sapiens
AYWPDVIHSFPNRS

SEQ ID NO: 14 - protein, 18 aa, Homo sapiens
QDQQLQNCTEPGEQPSPK

SEQ ID NO: 15 - protein, 12 aa, Homo sapiens
YFFNLSSMTCEK

SEQ ID NO: 16 - protein, 21 aa, Homo sapiens
KIPSFCYSPKDEGLCSANVTR

SEQ ID NO: 17 - protein, 24 aa, Homo sapiens
GRADDCALPYLGAICYCDLFCNRT

SEQ ID NO: 18 - protein, 14 aa, Homo sapiens
DSCKASCNCSNSIY

SEQ ID NO: 19 - protein, 19 aa, Homo sapiens
VIDFNCTTSSVSSALANTK

SEQ ID NO: 20 - protein, 30 aa, Homo sapiens
AKVAAANVSVTQPESTGDPNNMTLLAEEAR

SEQ ID NO: 21 - protein, 15 aa, Homo sapiens
TANDTSTEAYNLLLR

SEQ ID NO: 22 - protein, 14 aa, Homo sapiens
KYEQAKNISQDLEK

SEQ ID NO: 23 - protein, 14 aa, Homo sapiens
VNDNKTAAEEALRK

SEQ ID NO: 24 - protein, 37 aa, Homo sapiens
LSVDKDQYVEPENVTIQCDSGYGVVGPQSITCSGNRT

SEQ ID NO: 25 - protein, 25 aa, Homo sapiens
LPTQNITFQTESSVAEQEAEFQSPK

SEQ ID NO: 26 - protein, 22 aa, Homo sapiens
YRPENSVANDTGFTVVAPGKEK

SEQ ID NO: 27 - protein, 27 aa, Homo sapiens
NLDHGVLVVGYGFEGANSNNSKYWLVK

SEQ ID NO: 28 - protein, 32 aa, Homo sapiens
MLEAYNLTEKNFASVQGVSLESGSFPSYSAYR

SEQ ID NO: 29 - protein, 18 aa, Homo sapiens
DIKEAGNITTDGYEILGK

SEQ ID NO: 30 - protein, 17 aa, Homo sapiens
DVRPINVSSHCPSAGTK

SEQ ID NO: 31 - protein, 14 aa, Homo sapiens
VLQCLNISVLSQKR

SEQ ID NO: 32 - protein, 11 aa, Homo sapiens
EVIDTNLTTLR

SEQ ID NO: 33 - protein, 11 aa, Homo sapiens
ERFNISTPAFR

SEQ ID NO: 34 - protein, 13 aa, Homo sapiens
EGSLPGNSTISIR

SEQ ID NO: 35 - protein, 16 aa, Homo sapiens
QLEEIKRNASGDELVR

SEQ ID NO: 36 - protein, 13 aa, Homo sapiens
TFNLNTTEVEPCR

SEQ ID NO: 37 - protein, 31 aa, Homo sapiens
KLWADTTCGQNATELYCFYSENTDLTCRQPK

SEQ ID NO: 38 - protein, 19 aa, Homo sapiens
YFATNCSATFGLEDDVVKK

SEQ ID NO: 39 - protein, 26 aa, Homo sapiens
NKDIVTVANAVFVKNASEIEVPFVTR

SEQ ID NO: 40 - protein, 11 aa, Homo sapiens
NYSVNIREELK

SEQ ID NO: 41 - protein, 14 aa, Homo sapiens
QYLSYETLYANGSR

SEQ ID NO: 42 - protein, 19 aa, Homo sapiens
GANDSTSAMPEQMKFQWIR

SEQ ID NO: 43 - protein, 17 aa, Homo sapiens
SLSNLTEIHDQISGMAR

SEQ ID NO: 44 - protein, 28 aa, Homo sapiens
TRSCDSPAPQCGGWQCEGPGMEIANCSR

SEQ ID NO: 45 - protein, 16 aa, Homo sapiens
CGHLPRPNITQSCQLR

SEQ ID NO: 46 - protein, 14 aa, Homo sapiens
EVQCLSTNQTLSTR

SEQ ID NO: 47 - protein, 32 aa, Homo sapiens
NCGVNCSGDVFTALIGEIASPNYPKPYPENSR

SEQ ID NO: 48 - protein, 37 aa, Homo sapiens
YTCEEPYYYMENGGGGEYHCAGNGSWVNEVLGPELPK

SEQ ID NO: 49 - protein, 38 aa, Homo sapiens
LGPQVSLDPMKNVTCENGLPAVVSCVPGQVFSPDGPSR

SEQ ID NO: 50 - protein, 20 aa, Homo sapiens
HYHSMEVFTHYDLLNLNGTK

SEQ ID NO: 51 - protein, 13 aa, Homo sapiens
YYKGSLSYLNVTR

SEQ ID NO: 52 - protein, 14 aa, Homo sapiens
VLPVNVTDYCQLVR

SEQ ID NO: 53 - protein, 20 aa, Homo sapiens
NYYADNQTCDGELLFNMTKK

SEQ ID NO: 54 - protein, 27 aa, Homo sapiens
HYGPGWVSMANAGKDTNGSQFFITTVK

SEQ ID NO: 55 - protein, 17 aa, Homo sapiens
ISNSSDTVECECSENWK

SEQ ID NO: 56 - protein, 22 aa, Homo sapiens
AATCINPLNGSVCERPANHSAK

SEQ ID NO: 57 - protein, 17 aa, Homo sapiens
CINQSICEKCENLTTGK

SEQ ID NO: 58 - protein, 26 aa, Homo sapiens
CENLTTGKHCETCISGFYGDPTNGGK

SEQ ID NO: 59 - protein, 19 aa, Homo sapiens
NHPNITFFVYVSNFTWPIK

SEQ ID NO: 60 - protein, 21 aa, Homo sapiens
LRANQSWEDSNTDLVPAPAVR

SEQ ID NO: 61 - protein, 22 aa, Homo sapiens
SLTQGSLIVGDLAPVNGTSQGK

SEQ ID NO: 62 - protein, 32 aa, Homo sapiens
SQGLNLHTLLYLGGVEPSVPLSPATNMSAHFR

SEQ ID NO: 63 - protein, 34 aa, Homo sapiens
NLASRPYTFHSHGITYYKEHEGAIYPDNTTDFQR

SEQ ID NO: 64 - protein, 18 aa, Homo sapiens
AGLQAFFQVQECNKSSSK

SEQ ID NO: 65 - protein, 20 aa, Homo sapiens
ENLTAPGSDSAVFFEQGTTR

SEQ ID NO: 66 - protein, 25 aa, Homo sapiens
ELHHLQEQNVSNAFLDKGEFYIGSK

SEQ ID NO: 67 - protein, 30 aa, Homo sapiens
QLAHQSNSTNIFFSPVSIATAFAMLSLGTK

SEQ ID NO: 68 - protein, 32 aa, Homo sapiens
ADTHDEILEGLNFNLTEIPEAQIHEGFQELLR

SEQ ID NO: 69 - protein, 16 aa, Homo sapiens
YLGNATAIFFLPDEGK

SEQ ID NO: 70 - protein, 22 aa, Homo sapiens
AFTKNGSGAVFPVAGADVQTLR

SEQ ID NO: 71 - protein, 18 aa, Homo sapiens
LYQDEKAVLVNNITTGER

SEQ ID NO: 72 - protein, 17 aa, Homo sapiens
LQQDVLQFQKNQTNLER

SEQ ID NO: 73 - protein, 13 aa, Homo sapiens
ALGFENATQALGR

SEQ ID NO: 74 - protein, 11 aa, Homo sapiens
DAGVVCTNETR

SEQ ID NO: 75 - protein, 14 aa, Homo sapiens
YKGLNLTEDTYKPR

SEQ ID NO: 76 - protein, 13 aa, Homo sapiens
AAIPSALDTNSSK

SEQ ID NO: 77 - protein, 15 aa, Homo sapiens
TVIRPFYLTNSSGVD

SEQ ID NO: 78 - protein, 17 aa, Homo sapiens
AKFVGTPEVNQTTLYQR

SEQ ID NO: 79 - protein, 13 aa, Homo sapiens
SHNRSEEFLIAGK

SEQ ID NO: 80 - protein, 11 aa, Homo sapiens
NELMLNSSLMR

SEQ ID NO: 81 - protein, 16 aa, Homo sapiens
DPCSNVTCSFGSTCAR

SEQ ID NO: 82 - protein, 8 aa, Homo sapiens
DTFVNASR

SEQ ID NO: 83 - protein, 19 aa, Homo sapiens
VLSNNSDANLELINTWVAK

SEQ ID NO: 84 - protein, 21 aa, Homo sapiens
VGQLQLSHNLSLVILVPQNLK

SEQ ID NO: 85 - protein, 18 aa, Homo sapiens
IPCSQPPQIEHGTINSSR

SEQ ID NO: 86 - protein, 34 aa, Homo sapiens
WDPEVNCSMAQIQLCPPPPQIPNSHNMTTTLNYR

SEQ ID NO: 87 - protein, 28 aa, Homo sapiens
WQSIPLCVEKIPCSQPPQIEHGTINSSR

SEQ ID NO: 88 - protein, 13 aa, Homo sapiens
ISEENETTCYMGK

SEQ ID NO: 89 - protein, 13 aa, Homo sapiens
MDGASNVTCINSR

SEQ ID NO: 90 - protein, 29 aa, Homo sapiens
YQCRSPYEMFGDEEVMCLNGNWTEPPQCK

SEQ ID NO: 91 - protein, 15 aa, Homo sapiens
YNSQNQSNNQFVLYR

SEQ ID NO: 92 - protein, 23 aa, Homo sapiens
HGIQYFNNNTQHSSLFMLNEVKR

SEQ ID NO: 93 - protein, 12 aa, Homo sapiens
ITYSIVQTNCSK

SEQ ID NO: 94 - protein, 12 aa, Homo sapiens
LNAENNATFYFK

SEQ ID NO: 95 - protein, 23 aa, Homo sapiens
NNATVHEQVGGPSLTSDLQAQSK

SEQ ID NO: 96 - protein, 13 aa, Homo sapiens
CIQANYSLMENGK

SEQ ID NO: 97 - protein, 26 aa, Homo sapiens
ADGTVNQIEGEATPVNLTEPAKLEVK

SEQ ID NO: 98 - protein, 26 aa, Homo sapiens
GCVLLSYLNETVTVSASLESVRGNRS

SEQ ID NO: 99 - protein, 39 aa, Homo sapiens
GNEANYYSNATTDEHGLVQFSINTTNVMGTSLTVRVNYK

SEQ ID NO: 100 - protein, 33 aa, Homo sapiens
TEVSSNHVLIYLDKVSNQTLSLFFTVLQDVPVR

SEQ ID NO: 101 - protein, 11 aa, Homo sapiens
GLNVTLSSTGR

SEQ ID NO: 102 - protein, 32 aa, Homo sapiens
NNQIDHIDEKAFENVTDLQWLILDHNLLENSK

SEQ ID NO: 103 - protein, 18 aa, Homo sapiens
KLHINHNNLTESVGPLPK

SEQ ID NO: 104 - protein, 19 aa, Homo sapiens
LGSFEGLVNLTFIHLQHNR

SEQ ID NO: 105 - protein, 30 aa, Homo sapiens
LSHNELADSGIPGNSFNVSSLVELDLSYNK

SEQ ID NO: 106 - protein, 14 aa, Homo sapiens
GKEGHFYYNISEVK

SEQ ID NO: 107 - protein, 14 aa, Homo sapiens
MKVSNVSCQASVSR

SEQ ID NO: 108 - protein, 21 aa, Homo sapiens
IYSNHSALESLALIPLQAPLK

SEQ ID NO: 109 - protein, 35 aa, Homo sapiens
IYSNHSALESLALIPLQAPLKTMLQIGVMPMLNER

SEQ ID NO: 110 - protein, 14 aa, Homo sapiens
WFSAGLASNSSWLR

SEQ ID NO: 111 - protein, 19 aa, Homo sapiens
SVVAPATDGGLNLTSTFLR

SEQ ID NO: 112 - protein, 15 aa, Homo sapiens
EDALNETRESETKLK

SEQ ID NO: 113 - protein, 24 aa, Homo sapiens
LKELPGVCNETMMALWEECKPCLK

SEQ ID NO: 114 - protein, 20 aa, Homo sapiens
MLNTSSLLEQLNEQFNWVSR

SEQ ID NO: 115 - protein, 34 aa, Homo sapiens
MLNTSSLLEQLNEQFNWVSRLANLTQGEDQYYLR

SEQ ID NO: 116 - protein, 14 aa, Homo sapiens
LANLTQGEDQYYLR

SEQ ID NO: 117 - protein, 21 aa, Homo sapiens
SPYYNVSDEISFHCYDGYTLR

SEQ ID NO: 118 - protein, 16 aa, Homo sapiens
KVCQDCPLLAPLNDTR

SEQ ID NO: 119 - protein, 22 aa, Homo sapiens
AALAAFNAQNNGSNFQLEEISR

SEQ ID NO: 120 - protein, 28 aa, Homo sapiens
ANLTNFPENGTFVVNIAQLSQDDSGRYK

SEQ ID NO: 121 - protein, 23 aa, Homo sapiens
QIGLYPVLVIDSSGYVNPNYTGR

SEQ ID NO: 122 - protein, 22 aa, Homo sapiens
LSLLEEPGNGTFTVILNQLTSR

SEQ ID NO: 123 - protein, 23 aa, Homo sapiens
IIEGEPNLKVPGNVTAVLGETLK

SEQ ID NO: 124 - protein, 13 aa, Homo sapiens
SWPAVGNCSSALR

SEQ ID NO: 125 - protein, 22 aa, Homo sapiens
GHGHRNGTGHGNSTHHGPEYMR

SEQ ID NO: 126 - protein, 16 aa, Homo sapiens
ALPQPQNVTSLLGCTH

SEQ ID NO: 127 - protein, 16 aa, Homo sapiens
WFYIASAFRNEEYNKS

SEQ ID NO: 128 - protein, 24 aa, Homo sapiens
SVQEIQATFFYFTPNKTEDTIFLR

SEQ ID NO: 129 - protein, 15 aa, Homo sapiens
QDQCIYNTTYLNVQR

SEQ ID NO: 130 - protein, 27 aa, Homo sapiens
EYQTRQDQCIYNTTYLNVQRENGTISR

SEQ ID NO: 131 - protein, 22 aa, Homo sapiens
QDQCIYNTTYLNVQRENGTISR

SEQ ID NO: 132 - protein, 15 aa, Homo sapiens
QNQCFYNSSYLNVQR

SEQ ID NO: 133 - protein, 22 aa, Homo sapiens
QNQCFYNSSYLNVQRENGTVSR

SEQ ID NO: 134 - protein, 20 aa, Homo sapiens
FNLTETSEAEIHQSFQHLLR

SEQ ID NO: 135 - protein, 20 aa, Homo sapiens
TLNQSSDELQLSMGNAMFVK

SEQ ID NO: 136 - protein, 11 aa, Homo sapiens
LINDYVKNGTR

SEQ ID NO: 137 - protein, 29 aa, Homo sapiens
YTGASALFILPDQDKMEEVEAMLLPETLK

SEQ ID NO: 138 - protein, 13 aa, Homo sapiens
CGLVPVLAENYNK

SEQ ID NO: 139 - protein, 32 aa, Homo sapiens
CGLVPVLAENYNKSDNCEDTPEAGYFAVAVVK

SEQ ID NO: 140 - protein, 25 aa, Homo sapiens
QQQHLFGSNVTDCSGNFCLFRSETK

SEQ ID NO: 141 - protein, 18 aa, Homo sapiens
DIVEYYNDSNGSHVLQGR

SEQ ID NO: 142 - protein, 16 aa, Homo sapiens
FGCEIENNRSSGAFWK

SEQ ID NO: 143 - protein, 16 aa, Homo sapiens
LVGGPVAGGDPNQTIR

SEQ ID NO: 144 - protein, 14 aa, Homo sapiens
LNLTSPDLFWLVFR

SEQ ID NO: 145 - protein, 18 aa, Homo sapiens
VWQGHANASFCPHGYGCR

SEQ ID NO: 146 - protein, 11 aa, Homo sapiens
GINASSMAWAR

SEQ ID NO: 147 - protein, 16 aa, Homo sapiens
LHRLNASIADLQSQLR

SEQ ID NO: 148 - protein, 15 aa, Homo sapiens
GVHNASLALSASIGR

SEQ ID NO: 149 - protein, 16 aa, Homo sapiens
HTGNVVITNCSAAHSR

SEQ ID NO: 150 - protein, 17 aa, Homo sapiens
CLQHFYGPNHEHCFNRT

SEQ ID NO: 151 - protein, 21 aa, Homo sapiens
VLLGNESCTLTLSESTMNTLK

SEQ ID NO: 152 - protein, 19 aa, Homo sapiens
MVINVHEAGRNFTVACQHR

SEQ ID NO: 153 - protein, 19 aa, Homo sapiens
QHTVTTTTKGENFTETDVK

SEQ ID NO: 154 - protein, 11 aa, Homo sapiens
GLCVNASAVSR

SEQ ID NO: 155 - protein, 37 aa, Homo sapiens
LRAYLLPAPPAPGNASESEEDRSAGSVESPSVSSTHR

SEQ ID NO: 156 - protein, 21 aa, Homo sapiens
YKVDYESQSTDTQNFSSESKR

SEQ ID NO: 157 - protein, 22 aa, Homo sapiens
MYATIYELKEDKSYNVTSVLFR

SEQ ID NO: 158 - protein, 26 aa, Homo sapiens
TTLSGAPCQPWASEATYRNVTAEQAR

SEQ ID NO: 159 - protein, 23 aa, Homo sapiens
LGLSFNSISAVDNGSLANTPHLR

SEQ ID NO: 160 - protein, 17 aa, Homo sapiens
EQNYSDDVLANMISEPR

SEQ ID NO: 161 - protein, 12 aa, Homo sapiens
CRPINATLAVEK

SEQ ID NO: 162 - protein, 19 aa, Homo sapiens
NVFNETKNLLDKDWNIFSK

SEQ ID NO: 163 - protein, 20 aa, Homo sapiens
LRCDFEVLVVPWQNSSQLLK

SEQ ID NO: 164 - protein, 16 aa, Homo sapiens
HLLENSTASVSEAERK

SEQ ID NO: 165 - protein, 12 aa, Homo sapiens
YHYNGTFEDGKK

SEQ ID NO: 166 - protein, 18 aa, Homo sapiens
WGQNCSCHHGGYYNCEDK

SEQ ID NO: 167 - protein, 12 aa, Homo sapiens
FIRPFMQYNSTR

SEQ ID NO: 168 - protein, 42 aa, Homo sapiens
GTCEQGPSIVTPPKDIWNVTGAQVYLSCEVIGIPTPVLIWNK

SEQ ID NO: 169 - protein, 23 aa, Homo sapiens
VFPYISVMVNNGSLSYDHSKDGR

SEQ ID NO: 170 - protein, 19 aa, Homo sapiens
LCGPNVTDFPPFHANGTEK

SEQ ID NO: 171 - protein, 23 aa, Homo sapiens
DGYEPCVNEGMCVTYHNGTGYCK

SEQ ID NO: 172 - protein, 13 aa, Homo sapiens
HIGHANLTFEQLR

SEQ ID NO: 173 - protein, 19 aa, Homo sapiens
VEDEGNYTCLFVTFPQGSR

SEQ ID NO: 174 - protein, 33 aa, Homo sapiens
ENKDVLNFTCEPKSENYTYIWWLNGQSLPVSPR

SEQ ID NO: 175 - protein, 20 aa, Homo sapiens
ILILPNVTRNETGPYQCEIR

SEQ ID NO: 176 - protein, 23 aa, Homo sapiens
LHHHLDHNNTHHFHNDSITPSER

SEQ ID NO: 177 - protein, 20 aa, Homo sapiens
CLIQELCQCRPGEGNCSCCK

SEQ ID NO: 178 - protein, 17 aa, Homo sapiens
LSSSNASVAWMPGADGR

SEQ ID NO: 179 - protein, 21 aa, Homo sapiens
YFQNYSYGGVIQDDHIPFLRR

SEQ ID NO: 180 - protein, 11 aa, Homo sapiens
GLPGGNASLPR

SEQ ID NO: 181 - protein, 15 aa, Homo sapiens
DLGPTLANSTHHNVR

SEQ ID NO: 182 - protein, 19 aa, Homo sapiens
VASVININPNTTHSTGSCR

SEQ ID NO: 183 - protein, 28 aa, Homo sapiens
LGVSSVPSCYLIYPNGSHGLINVVKPLR

SEQ ID NO: 184 - protein, 21 aa, Homo sapiens
FTFTSHTPGDHQICLHSNSTR

SEQ ID NO: 185 - protein, 13 aa, Homo sapiens
FKIGYSNNGSDWK

SEQ ID NO: 186 - protein, 28 aa, Homo sapiens
RGQFESVAPSQNFSDSSESDTHPFVIAK

SEQ ID NO: 187 - protein, 26 aa, Homo sapiens
WVLTAAHCLLYPPWDKNFTENDLLVR

SEQ ID NO: 188 - protein, 17 aa, Homo sapiens
RHEEGHMLNCTCFGQGR

SEQ ID NO: 189 - protein, 10 aa, Homo sapiens
GWNWTSGFNK

SEQ ID NO: 190 - protein, 10 aa, Homo sapiens
GIGNYSCSYR

SEQ ID NO: 191 - protein, 30 aa, Homo sapiens
TAGWNVPIGTLRPFLNWTGPPEPIEAAVAR

SEQ ID NO: 192 - protein, 33 aa, Homo sapiens
AHLNVSGIPCSVLLADVEDLIQQQISNDTVSPR

SEQ ID NO: 193 - protein, 21 aa, Homo sapiens
IVGGTNSSWGEWPWQVSLQVK

SEQ ID NO: 194 - protein, 8 aa, Homo sapiens
HMPWNITR

SEQ ID NO: 195 - protein, 23 aa, Homo sapiens
HSNCTHQQDAGVTCSDGSNLEMR

SEQ ID NO: 196 - protein, 21 aa, Homo sapiens
TVLTPATNHMGNVTFTIPANR

SEQ ID NO: 197 - protein, 12 aa, Homo sapiens
NTSVGLLYSGCR

SEQ ID NO: 198 - protein, 18 aa, Homo sapiens
KFNITESVLQGLLKPLFK

SEQ ID NO: 199 - protein, 12 aa, Homo sapiens
NGTQLQNFTLDR

SEQ ID NO: 200 - protein, 15 aa, Homo sapiens
GVFITNETGQPLIGK

SEQ ID NO: 201 - protein, 17 aa, Homo sapiens
DHLLLPATTHNKTTRPK

SEQ ID NO: 202 - protein, 12 aa, Homo sapiens
LNGSITSPGWPK

SEQ ID NO: 203 - protein, 17 aa, Homo sapiens
DHLLLPAISHNKTSRPK

SEQ ID NO: 204 - protein, 14 aa, Homo sapiens
NATSYPPMCTQDPK

SEQ ID NO: 205 - protein, 11 aa, Homo sapiens
RFANEYPNITR

SEQ ID NO: 206 - protein, 22 aa, Homo sapiens
HIWSLEISNKPNVSEPEEPKIR

SEQ ID NO: 207 - protein, 18 aa, Homo sapiens
GKDLDTDFTNNASQPETK

SEQ ID NO: 208 - protein, 29 aa, Homo sapiens
ELLIFLAQYLCNEYQKGNETIVNLIHSTR

SEQ ID NO: 209 - protein, 9 aa, Homo sapiens
NFVITYNRT

SEQ ID NO: 210 - protein, 17 aa, Homo sapiens
YSVANDTGFVDIPKQEK

SEQ ID NO: 211 - protein, 35 aa, Homo sapiens
QLVHSFAEGQDQGSAYANRTALFPDLLAQGNASLR

SEQ ID NO: 212 - protein, 14 aa, Homo sapiens
VVLGANGTYSCLVR

SEQ ID NO: 213 - protein, 25 aa, Homo sapiens
DQGSAYANRTALFPDLLAQGNASLR

SEQ ID NO: 214 - protein, 13 aa, Homo sapiens
QINSSISGNLWDK

SEQ ID NO: 215 - protein, 8 aa, Homo sapiens
FLSYNVTR

SEQ ID NO: 216 - protein, 8 aa, Homo sapiens
NNTFLSLR

SEQ ID NO: 217 - protein, 16 aa, Homo sapiens
TCPAGVMGENNTLVWK

SEQ ID NO: 218 - protein, 24 aa, Homo sapiens
YLETLPNTSIIIPFHNEGWSSLLR

SEQ ID NO: 219 - protein, 12 aa, Homo sapiens
FISPFTQFNITR

SEQ ID NO: 220 - protein, 15 aa, Homo sapiens
LSPQINASNFYFNKT

SEQ ID NO: 221 - protein, 31 aa, Homo sapiens
YGCCPMPNATCCSDHLHCCPQDTVCDLIQSK

SEQ ID NO: 222 - protein, 12 aa, Homo sapiens
TSIPTINMENKT

SEQ ID NO: 223 - protein, 25 aa, Homo sapiens
MFLLVGAPKANTTQPGIVEGGQVLK

SEQ ID NO: 224 - protein, 12 aa, Homo sapiens
LSCAFKTENQTR

SEQ ID NO: 225 - protein, 8 aa, Homo sapiens
LALNLTLR

SEQ ID NO: 226 - protein, 20 aa, Homo sapiens
RANTSALAVPSPVSNSASAR

SEQ ID NO: 227 - protein, 17 aa, Homo sapiens
LRNATASLWSGPGLEDR

SEQ ID NO: 228 - protein, 14 aa, Homo sapiens
ANKWTGHNVTVVQR

SEQ ID NO: 229 - protein, 41 aa, Homo sapiens
LLNKFIVESLTPSSLSLMHSPPGTQNISEINLSPMEISTFR

SEQ ID NO: 230 - protein, 13 aa, Homo sapiens
RPYVSYVNNSIAR

SEQ ID NO: 231 - protein, 11 aa, Homo sapiens
WVSLDNWTYSK

SEQ ID NO: 232 - protein, 14 aa, Homo sapiens
ALLTNVSSVALGSR

SEQ ID NO: 233 - protein, 32 aa, Homo sapiens
TGYTMDNMTGLCRPVCAQGCVNGSCVEPDHCR

SEQ ID NO: 234 - protein, 30 aa, Homo sapiens
LLKINSTADLDFIQQAISYSSFPFWMGLSR

SEQ ID NO: 235 - protein, 27 aa, Homo sapiens
HTGPGILSMANAGPNTNGSQFFICTAK

SEQ ID NO: 236 - protein, 9 aa, Homo sapiens
LSGNLTLLR

SEQ ID NO: 237 - protein, 12 aa, Homo sapiens
ILRNVSECFLAR

SEQ ID NO: 238 - protein, 18 aa, Homo sapiens
IHVAGETDSSNLNVSEPR

SEQ ID NO: 239 - protein, 12 aa, Homo sapiens
FLRYNCSIESPR

SEQ ID NO: 240 - protein, 15 aa, Homo sapiens
RIAVDWESLGYNITR

SEQ ID NO: 241 - protein, 18 aa, Homo sapiens
APQHVVNHLPPYTNVSLK

SEQ ID NO: 242 - protein, 11 aa, Homo sapiens
FSADLGYNGTR

SEQ ID NO: 243 - protein, 8 aa, Homo sapiens
LIQPWNRT

SEQ ID NO: 244 - protein, 21 aa, Homo sapiens
TGVHDADFESNVTATLASINK

SEQ ID NO: 245 - protein, 11 aa, Homo sapiens
KNITDLVEGAK

SEQ ID NO: 246 - protein, 14 aa, Homo sapiens
SVNPNDTCLASCVK

SEQ ID NO: 247 - protein, 13 aa, Homo sapiens
NTTEVVNTMCGYK

SEQ ID NO: 248 - protein, 11 aa, Homo sapiens
HNFTASLLTLR

SEQ ID NO: 249 - protein, 18 aa, Homo sapiens
VLSSIQEGTVPDNTSSAR

SEQ ID NO: 250 - protein, 18 aa, Homo sapiens
NYTLTGRDSCTLPASAEK

SEQ ID NO: 251 - protein, 28 aa, Homo sapiens
TGVCPELQADQNCTQECVSDSECADNLK

SEQ ID NO: 252 - protein, 22 aa, Homo sapiens
DYNSSREDSLQDAWDYVQAQVK

SEQ ID NO: 253 - protein, 8 aa, Homo sapiens
QVYNLTVR

SEQ ID NO: 254 - protein, 14 aa, Homo sapiens
YAESDPTVPCNETR

SEQ ID NO: 255 - protein, 19 aa, Homo sapiens
LSWAKPQPLNETAPSNLWK

SEQ ID NO: 256 - protein, 21 aa, Homo sapiens
ADANPPATEYHWTTLNGSLPK

SEQ ID NO: 257 - protein, 20 aa, Homo sapiens
DIENLKDASSFLAEWQNITK

SEQ ID NO: 258 - protein, 23 aa, Homo sapiens
SLVTQYLNATGNRWCSWSLSQAR

SEQ ID NO: 259 - protein, 20 aa, Homo sapiens
KLGDCISEDSYPDGNITWYR

SEQ ID NO: 260 - protein, 12 aa, Homo sapiens
NAIKEGDNITLK

SEQ ID NO: 261 - protein, 12 aa, Homo sapiens
NATVVWMKDNIR

SEQ ID NO: 262 - protein, 21 aa, Homo sapiens
IIISPEENVTLTCTAENQLER

SEQ ID NO: 263 - protein, 34 aa, Homo sapiens
AFSNSSYVLNPTTGELVFDPLSASDTGEYSCEAR

SEQ ID NO: 264 - protein, 18 aa, Homo sapiens
ILLTCSLNDSATEVTGHR

SEQ ID NO: 265 - protein, 16 aa, Homo sapiens
ITDSEDKALMNGSESR

SEQ ID NO: 266 - protein, 18 aa, Homo sapiens
QQMENYPKNNHTASILDR

SEQ ID NO: 267 - protein, 18 aa, Homo sapiens
CCGAANYTDWEKIPSMSK

SEQ ID NO: 268 - protein, 18 aa, Homo sapiens
LRNPCTSEQNCTSPFSYK

SEQ ID NO: 269 - protein, 20 aa, Homo sapiens
FKLEWLGNCSGLNDETYGYK

SEQ ID NO: 270 - protein, 21 aa, Homo sapiens
VLGFKPKPPKNESLETYPVMK

SEQ ID NO: 271 - protein, 18 aa, Homo sapiens
ANIQFGDNGTTISAVSNK

SEQ ID NO: 272 - protein, 19 aa, Homo sapiens
HRPTAGAFNHSDLDAELRR

SEQ ID NO: 273 - protein, 9 aa, Homo sapiens
TLTLFNVTR

SEQ ID NO: 274 - protein, 15 aa, Homo sapiens
HKYLWSEPQNCSATK

SEQ ID NO: 275 - protein, 12 aa, Homo sapiens
HIPGLIHNMTAR

SEQ ID NO: 276 - protein, 12 aa, Homo sapiens
FLNNGTCTAEGK

SEQ ID NO: 277 - protein, 19 aa, Homo sapiens
FKLSDLSINSTECLHVHCR

SEQ ID NO: 278 - protein, 26 aa, Homo sapiens
SIPACVPWSPYLFQPNDTCIVSGWGR

SEQ ID NO: 279 - protein, 20 aa, Homo sapiens
FNTTYINIGSSYFPEHGYFR

SEQ ID NO: 280 - protein, 15 aa, Homo sapiens
VNLSFDFPFYGHFLR

SEQ ID NO: 281 - protein, 15 aa, Homo sapiens
FCRDNYTDLVAIQNK

SEQ ID NO: 282 - protein, 13 aa, Homo sapiens
IGGIWTWVGTNKS

SEQ ID NO: 283 - protein, 38 aa, Homo sapiens
VAYLDPLELSEGKVLSLPLNSSAVVNCSVHGLPTPALR

SEQ ID NO: 284 - protein, 21 aa, Homo sapiens
SLHVPGLNKTSSFSCEAHNAK

SEQ ID NO: 285 - protein, 14 aa, Homo sapiens
EHVHNASAHLTFNK

SEQ ID NO: 286 - protein, 13 aa, Homo sapiens
LYANHTSLPASAR

SEQ ID NO: 287 - protein, 24 aa, Homo sapiens
SRNPGSSCIGADPNRNWNASFAGK

SEQ ID NO: 288 - protein, 40 aa, Homo sapiens
RNDAGSYECEIQNPASANRSDPVTLNVLYGPDGPTISPSK

SEQ ID NO: 289 - protein, 9 aa, Homo sapiens
QTLFFNGTR

SEQ ID NO: 290 - protein, 24 aa, Homo sapiens
SACNCTAGAACDAVNGSCLCPAGR

SEQ ID NO: 291 - protein, 16 aa, Homo sapiens
SPHRPILQAGLPANKT

SEQ ID NO: 292 - protein, 21 aa, Homo sapiens
HIEVNGSKIGPDNLPYVQILK

SEQ ID NO: 293 - protein, 29 aa, Homo sapiens
NCQDIDECVTGIHNCSINETCFNIQGGFR

SEQ ID NO: 294 - protein, 10 aa, Homo sapiens
ILSNLSCNVK

SEQ ID NO: 295 - protein, 33 aa, Homo sapiens
EANEVANQILNLTADGQNLTSANITNIVEQVKR

SEQ ID NO: 296 - protein, 11 aa, Homo sapiens
LFNVTPQDEQK

SEQ ID NO: 297 - protein, 14 aa, Homo sapiens
IITVSTNGSIHSPR

SEQ ID NO: 298 - protein, 14 aa, Homo sapiens
YTLDPNITSAGPTK

SEQ ID NO: 299 - protein, 33 aa, Homo sapiens
SLPCDICKDVVTAAGDMLKDNATEEEILVYLEK

SEQ ID NO: 300 - protein, 15 aa, Homo sapiens
TCDWLPKPNMSASCK

SEQ ID NO: 301 - protein, 13 aa, Homo sapiens
NSTKQEILAALEK

SEQ ID NO: 302 - protein, 15 aa, Homo sapiens
VYLFDFPEGKNASVR

SEQ ID NO: 303 - protein, 19 aa, Homo sapiens
HPSCWNLVNGTVVPLGEMR

SEQ ID NO: 304 - protein, 21 aa, Homo sapiens
RPPARPGPLSTANHTALRGSH

SEQ ID NO: 305 - protein, 22 aa, Homo sapiens
VAQPGINYALGTNVSYPNNLLR

SEQ ID NO: 306 - protein, 31 aa, Homo sapiens
NELVQLYQVGEVRPFYYGLCTPCQAPTNYSR

SEQ ID NO: 307 - protein, 16 aa, Homo sapiens
ESWGQESNAGNQTVVR

SEQ ID NO: 308 - protein, 15 aa, Homo sapiens
LNPTVTYGNDSFSAK

SEQ ID NO: 309 - protein, 24 aa, Homo sapiens
MVSHHNLTTGATLINEQWLLTTAK

SEQ ID NO: 310 - protein, 26 aa, Homo sapiens
NLFLNHSENATAKDIAPTLTLYVGKK

SEQ ID NO: 311 - protein, 20 aa, Homo sapiens
SGPKNMTFDLPSDATVVLNR

SEQ ID NO: 312 - protein, 21 aa, Homo sapiens
SGPKNMTFDLPSDATVVLNRS

SEQ ID NO: 313 - protein, 24 aa, Homo sapiens
YSVQLMSFVYNLSDTHLFPNASSK

SEQ ID NO: 314 - protein, 19 aa, Homo sapiens
VDKDLQSLEDILHQVENKT

SEQ ID NO: 315 - protein, 14 aa, Homo sapiens
KLPPGLLANFTLLR

SEQ ID NO: 316 - protein, 23 aa, Homo sapiens
YGEEYGNLTRPDITFTYFQPKPR

SEQ ID NO: 317 - protein, 9 aa, Homo sapiens
WSDIWNATK

SEQ ID NO: 318 - protein, 29 aa, Homo sapiens
RNPPMGGNVVIFDTVITNQEEPYQNHSGR

SEQ ID NO: 319 - protein, 19 aa, Homo sapiens
VCLANGSWSGATPDCVPVR

SEQ ID NO: 320 - protein, 18 aa, Homo sapiens
CLSNGSWSGSSPSCLPCR

SEQ ID NO: 321 - protein, 18 aa, Homo sapiens
SLPNFPNTSATANATGGR

SEQ ID NO: 322 - protein, 33 aa, Homo sapiens
EGDHEFLEVPEAQEDVEATFPVHQPGNYSCSYR

SEQ ID NO: 323 - protein, 13 aa, Homo sapiens
VYKPSAGNNSLYR

SEQ ID NO: 324 - protein, 34 aa, Homo sapiens
DTAVFECLPQHAMFGNDTITCTTHGNWTKLPECR

SEQ ID NO: 325 - protein, 11 aa, Homo sapiens
LGNWSAMPSCK

SEQ ID NO: 326 - protein, 17 aa, Homo sapiens
FSDGLESNSSTQFEVKK

SEQ ID NO: 327 - protein, 14 aa, Homo sapiens
LAFQNMNGSEYFVK

SEQ ID NO: 328 - protein, 13 aa, Homo sapiens
LQNLTLPTNASIK

SEQ ID NO: 329 - protein, 16 aa, Homo sapiens
FNPGAESVVLSNSTLK

SEQ ID NO: 330 - protein, 23 aa, Homo sapiens
LGACNDTLQQLMEVFKFDTISEK

SEQ ID NO: 331 - protein, 14 aa, Homo sapiens
AAINKWVSNKTEGR

SEQ ID NO: 332 - protein, 20 aa, Homo sapiens
GTAGNALMDGASQLMGENRT

SEQ ID NO: 333 - protein, 15 aa, Homo sapiens
GYYNQSEDGSHTLQR

SEQ ID NO: 334 - protein, 16 aa, Homo sapiens
LSALDNLLNHSSMFLK

SEQ ID NO: 335 - protein, 18 aa, Homo sapiens
VINETWAWKNATLAEQAK

SEQ ID NO: 336 - protein, 21 aa, Homo sapiens
DKNGTRAEPPLNASASDQGEK

SEQ ID NO: 337 - protein, 20 aa, Homo sapiens
NTTWQAGHNFYNVDMSYLKR

SEQ ID NO: 338 - protein, 24 aa, Homo sapiens
SIVYSCEWPLYMWPFQKPNYTEIR

SEQ ID NO: 339 - protein, 20 aa, Homo sapiens
GYVEGVHNSSIALSDCFGLR

SEQ ID NO: 340 - protein, 27 aa, Homo sapiens
GLLHLENASYGIEPLQNSSHFEHIIYR

SEQ ID NO: 341 - protein, 26 aa, Homo sapiens
LGQAPANWYNDTYPLSPPQRTPAGIR

SEQ ID NO: 342 - protein, 17 aa, Homo sapiens
KCATVTENATGDLATSR

SEQ ID NO: 343 - protein, 19 aa, Homo sapiens
FLSSSPHLPPSSYFNASGR

SEQ ID NO: 344 - protein, 16 aa, Homo sapiens
LPEMAQPVDPAHNVSR

SEQ ID NO: 345 - protein, 17 aa, Homo sapiens
IYVLDGTQNDTAFVFPR

SEQ ID NO: 346 - protein, 14 aa, Homo sapiens
HYENHSAIMLGIKK

SEQ ID NO: 347 - protein, 12 aa, Homo sapiens
LVQLFPNDTSLK

SEQ ID NO: 348 - protein, 11 aa, Homo sapiens
LSFGSNSSDFK

SEQ ID NO: 349 - protein, 19 aa, Homo sapiens
VNVVNSTLAEVHWDPVPLK

SEQ ID NO: 350 - protein, 11 aa, Homo sapiens
NNTTFLECAPK

SEQ ID NO: 351 - protein, 11 aa, Homo sapiens
SDSGNYTCLVR

SEQ ID NO: 352 - protein, 11 aa, Homo sapiens
LENITNPWSPR

SEQ ID NO: 353 - protein, 10 aa, Homo sapiens
WNLSASDITR

SEQ ID NO: 354 - protein, 24 aa, Homo sapiens
HVSFLPAPRPVVNVSGGPLLYSHR

SEQ ID NO: 355 - protein, 22 aa, Homo sapiens
AECCASGNIDTAWSNLTHPGNK

SEQ ID NO: 356 - protein, 32 aa, Homo sapiens
LNVEAANWTVRGEEDFSWFGYSLHGVTVDNRT

SEQ ID NO: 357 - protein, 13 aa, Homo sapiens
LQNNENNISCVER

SEQ ID NO: 358 - protein, 11 aa, Homo sapiens
GRPNITHAVPK

SEQ ID NO: 359 - protein, 19 aa, Homo sapiens
LNSTHCQDINECAMPGVCR

SEQ ID NO: 360 - protein, 20 aa, Homo sapiens
SHTNTSHVMQYGNKTISTMK

SEQ ID NO: 361 - protein, 21 aa, Homo sapiens
SQILEGLGFNLTELSESDVHR

SEQ ID NO: 362 - protein, 10 aa, Homo sapiens
LTFYGNWSEK

SEQ ID NO: 363 - protein, 13 aa, Homo sapiens
VHINTTSDSILLK

SEQ ID NO: 364 - protein, 21 aa, Homo sapiens
LGLGNNKITDIENGSLANIPR

SEQ ID NO: 365 - protein, 11 aa, Homo sapiens
QVQEQCPNITR

SEQ ID NO: 366 - protein, 16 aa, Homo sapiens
AFGIVECVLCTCNVTK

SEQ ID NO: 367 - protein, 19 aa, Homo sapiens
IIVPLNNRENISDPTSPLR

SEQ ID NO: 368 - protein, 10 aa, Homo sapiens
YVQNGTYTVK

SEQ ID NO: 369 - protein, 10 aa, Homo sapiens
LHEITNETFR

SEQ ID NO: 370 - protein, 22 aa, Homo sapiens
VTQVYAENGTVLQGSTVASVYK

SEQ ID NO: 371 - protein, 37 aa, Homo sapiens
ILTNNSQTPILSPQEVVSCSQYAQGCEGGFPYLIAGK

SEQ ID NO: 372 - protein, 8 aa, Homo sapiens
NIETNYTR

SEQ ID NO: 373 - protein, 12 aa, Homo sapiens
SPVQENSSDLNK

SEQ ID NO: 374 - protein, 20 aa, Homo sapiens
RVEYGAEGRHNSTIDVGGQK

SEQ ID NO: 375 - protein, 13 aa, Homo sapiens
NIQNNVSCYLEGK

SEQ ID NO: 376 - protein, 17 aa, Homo sapiens
IIYGVENSSTFLECSPK

SEQ ID NO: 377 - protein, 21 aa, Homo sapiens
AKLDAFFALEGFPTEPNSSSR

SEQ ID NO: 378 - protein, 15 aa, Homo sapiens
GWAPPDKSIINATDP

SEQ ID NO: 379 - protein, 14 aa, Homo sapiens
YMQNLTVEQPIEVK

SEQ ID NO: 380 - protein, 21 aa, Homo sapiens
VVGVPYQGNATALFILPSEGK

SEQ ID NO: 381 - protein, 18 aa, Homo sapiens
IFLKEVNDTLLVNELKSK

SEQ ID NO: 382 - protein, 15 aa, Homo sapiens
QGEHLSNSTSAFSTR

SEQ ID NO: 383 - protein, 20 aa, Homo sapiens
ILILPSVTRNETGPYQCEIR

SEQ ID NO: 384 - protein, 18 aa, Homo sapiens
TMLVQKNVTSESTCCVAK

SEQ ID NO: 385 - protein, 17 aa, Homo sapiens
DGTPLSDGQSNHTVSSK

SEQ ID NO: 386 - protein, 13 aa, Homo sapiens
KVEAEALNATAIR

SEQ ID NO: 387 - protein, 12 aa, Homo sapiens
LSDTANYTCVAK

SEQ ID NO: 388 - protein, 21 aa, Homo sapiens
NATSVDSGAPGGAAPGGPGFR